(19) United States
(12) Patent Application Publication
(10) Pub. No.: US 2005/0079202 A1
(43) Pub. Date: Apr. 14, 2005

(54) Implantable elastomeric depot compositions and uses thereof

(76) Inventors: Guohua Chen, Sunnyvale, CA (US); Paul R. Houston, Hayward, CA (US); Lothar W. Kleiner, Los Altos, CA (US); Aruna Nathan, Bridgewater, NJ (US); Joel Rosenblatt, Watchung, NJ (US)

Correspondence Address: TRASK BRITT, P.O. BOX 2550, SALT LAKE CITY, UT, 84110, US

(21) Appl. No.: 10/857,609

(22) Filed: May 28, 2004

Related U.S. Application Data
(60) Provisional application No. 60/474,874, filed on May 30, 2003.

(51) Int. Cl. A61F 2/00

(52) U.S. Cl. 424/426

(57) ABSTRACT

Methods and compositions for systemically or locally administering a beneficial agent to a subject are described, and include, for example, implantable elastomeric depot compositions that can be injected into a desired location and which can provide controlled release of a beneficial agent over a prolonged duration of time. The compositions include a biocompatible, elastomeric polymer, a biocompatible solvent having low water miscibility that forms an elastomeric viscous gel with the polymer and limits water uptake by the implant, and a beneficial agent.

### FIELD OF THE INVENTION

The present invention relates to an implantable elastomeric depot composition that can be injected into a desired location and which can provide controlled release of a beneficial agent over a specified/desired duration of time. The present invention also relates to a method of preparing and administering the composition.

### BACKGROUND OF THE INVENTION

Description of the Related Art: Biodegradable polymers have been used for many years in medical applications. Illustrative devices composed of the biodegradable polymers include sutures, surgical clips, staples, implants, and drug delivery systems. The majority of these biodegradable polymers have been based upon glycolide, lactide, caprolactone, p-dioxanone (PDO), trimethylene carbonate (TMC), poly(propylene fumarate), poly(orthoesters), polyphosphoester and copolymers thereof.

Use of biodegradable elastomeric polymers for medical purposes is well established. (See, e.g., U.S. Pat. Nos. 6,113,624; 5,868,788; 5,714,551; 5,713,920; 5,639,851 and 5,468,253.) However, these materials do not always satisfy the demand for a biodegradable implant. For example, while elastomeric polymers possess the requisite biocompatability, strength and processability, for numerous medical device applications, such elastomeric polymers are not bioabsorbable in bodily tissue, potentially resulting in adverse tissue reaction or other complications associated with the occurrence of foreign matter in bodily tissue. There is a need for bioabsorbable elastomeric polymers that exhibit a desirable degree of elasticity necessary for use in implantable depot drug delivery systems.

The biodegradable polymers can be thermoplastic materials, meaning that they can be heated and formed into various shapes, such as fibers, clips, staples, pins, films, etc. Alternatively, they can be thermosetting materials formed by cross-linking reactions, which lead to high molecular weight materials that do not melt or form flowable liquids at high temperatures. Although elastomeric, thermoplastic and thermosetting biodegradable polymers have many useful biomedical applications, there are several important limitations to their use in the bodies of various animals, including humans, animals, birds, fish, and reptiles.

Solid implant drug delivery systems containing a drug incorporated in thermoplastic or thermosetting biodegradable polymers have been widely used. Such implants have to be inserted into the body through an incision which is sometimes larger than that desired by the medical professional and occasionally lead to a reluctance of the patients to accept such an implant or drug delivery system. The following U.S. Pat. Nos. 6,113,624; 5,868,788; 5,714,551; 5,713,920; 5,639,851; 5,468,253; 5,456,679; 5,336,057; 5,308,348; 5,279,608; 5,234,693; 5,234,692; 5,209,746; 5,151,093; 5,137,727; 5,112,614; 5,085,866; 5,059,423; 5,057,318; 4,865,845; 4,008,719; 3,987,790 and 3,797,492 are believed to be representative of such drug delivery systems and are incorporated herein by reference. These patents disclose reservoir devices, osmotic delivery devices and pulsatile delivery devices for delivering beneficial agents.

Injecting drug delivery systems as small particles, microspheres, or microcapsules avoids the incision needed to implant drug delivery systems. However, these materials do not always satisfy the demand for a biodegradable implant. These materials are particulate in nature, do not form a continuous film or solid implant with the structural integrity needed for certain prostheses, the particles tend to aggregate and thus their behavior is hard to predict. When inserted into certain body cavities, such as a mouth, a periodontal pocket, the eye, or the vagina, where there is considerable fluid flow, these small particles, microspheres, or microcapsules are poorly retained because of their small size and discontinuous nature. Further, if there are complications, removal of microcapsule or small-particle systems from the body without extensive surgical intervention is considerably more difficult than with solid implants. Additionally, manufacture, storage and injectability of microspheres or microcapsules prepared from these polymers and containing drugs for release into the body present problems.

The art has developed various drug delivery systems in response to the aforementioned challenges. The following U.S. Pat. Nos. 6,432,438; 5,990,194; 5,780,044; 5,733,950; 5,620,700; 5,599,552; 5,556,905 5,278,201; 5,242,910 and 4,938,763; and PCT publications WO 98/27962; WO 02/00137 and WO 02/058670 are believed to be representative and are incorporated herein by reference. See also Jain, R. et al., “Controlled drug delivery by biodegradable poly(ester) devices: different preparative approaches,” Drug Dev. Ind. Pharm., 24(8): 703-727, 1998; Eliaz, R. E. and Kost, J., “Characterization of a polymeric PLGA-injectable implant deliver system for the controlled release of proteins,” J. Biomed. Master Res., 50(3): 388-396, 2000; and Jain, R. A., “The manufacturing techniques of various drug loaded biodegradable poly(lactide-co-glycolide) (PLGA) devices,” Biomaterials, 21(23): 2475-90, 2000. These patents and publications disclose polymer compositions for injectable implants using solvents and/or plasticizers.

Previously described polymer compositions for injectable implants have used solvent/plasticizers that are very or relatively soluble in aqueous body fluids to promote rapid solidification of the polymer at the implant site and promote diffusion of drug from the implant. Rapid migration of water into such polymeric implants utilizing water soluble polymer solvents when the implants are placed in the body and exposed to aqueous body fluids presents a serious problem. The rapid water uptake often results in implants having pore structures that are non-homogeneous in size and shape. Typically, the surface pores take on a finger-like pore structure extending for as much as one-third of a millimeter or more from the implant surface into the implant, and such finger-like pores are open at the surface of the implant to the environment of use. The internal pores tend to be smaller and less accessible to the fluids present in the environment of use. The rapid water uptake characteristic often results in uncontrolled release of beneficial agent that is manifested by an initial, rapid release of beneficial agent from the polymer composition, corresponding to a “burst” of beneficial agent being released from the implant. The burst often results in a substantial portion of the beneficial agent, if not all, being released in a very short time, e.g., hours or one to two days. Such an effect can be unacceptable, particularly in those circumstances where a controlled delivery is desired, i.e., delivery of beneficial agent in a controlled manner over a period of greater than or equal to a week and up to one year, or where there is a narrow therapeutic window and release of excess beneficial agent can result in adverse consequences to the subject being treated, or where it is necessary to mimic the naturally occurring daily profile of beneficial agents, such as hormones and the like, in the body of the subject being treated.

Accordingly, when such devices are implanted, the finger-like pores allow very rapid uptake of aqueous body fluids into the interior of the implant with consequent immediate and rapid dissolution of significant quantities of beneficial agent and unimpeded diffusion of beneficial agent into the environment of use, producing the burst effect discussed above.

Furthermore, rapid water uptake can result in premature polymer precipitation such that a hardened implant or one with a hardened skin is produced. The inner pores and much of the interior of the polymer containing beneficial agent are shut off from contact with the body fluids and a significant reduction in the release of beneficial agent can result over a not insignificant period of time (“lag time”). That lag time is undesirable from the standpoint of presenting a controlled, sustained release of beneficial agent to the subject being treated. What one observes, then, is a burst of beneficial agent being released in a short time period immediately after implantation, a lag time in which no or very little beneficial agent is being released, and subsequently continued delivery of beneficial agent (assuming beneficial agent remains after the burst) until the supply of beneficial agent is exhausted.

Various approaches to control burst and modulate and stabilize the delivery of the beneficial agent have been described. The following U.S. Pat. Nos. 6,130,200; 5,990,194; 5,780,044; 5,733,950; 5,656,297; 5,654,010; 4,985,404 and 4,853,218 and PCT publication WO 98/27962 are believed to be representative and are incorporated herein by reference. Notwithstanding some success, those methods have not been entirely satisfactory for the large number of beneficial agents that would be effectively delivered by implants. There is a need for elastomeric implantable depot compositions having a desirable degree of elasticity while providing a controlled sustained delivery of beneficial agents.

### SUMMARY OF THE INVENTION

The present invention provides an implantable elastomeric depot composition and a method of using the implantable elastomeric depot composition for systemic and local administration of a beneficial agent to a subject over a prolonged duration of time. In particular, the invention provides an implantable elastomeric depot composition with desired elasticity while providing for controlled release of the beneficial agent to the subject being treated, the release being controlled over a period greater than or equal to one week and up to one year after administration, preferably over a period equal to or greater than two weeks after administration, more preferably greater than one month, even more preferably about two months to about three months, and most preferably about three months to about six months after administration. A single administration of the implantable elastomeric depot composition provides longer sustained release of active agents over a prolonged duration of time, thus reducing the frequency of administration and improving patient compliance. Additionally, the invention provides a method of preparing the implantable elastomeric depot composition. In preferred embodiments, the implantable elastomeric depot composition is an implantable elastomeric depot composition.

In one aspect, the invention pertains to an implantable elastomeric depot composition for sustained delivery of a beneficial agent to a subject in a controlled manner over a predetermined duration of time after administration, comprising: (a) an elastomeric viscous gel formulation comprising: (1) a bioerodible, biocompatible polymer, wherein the polymer is an elastomeric polymer; and (2) a solvent having a miscibility in water of less than or equal to 7 wt. % at 25° C., in an amount effective to plasticize the polymer and form a gel therewith; and (b) a beneficial agent dissolved or dispersed in the gel, wherein the beneficial agent is delivered over a duration equal to or greater than one month. Preferably, the polymer is a lactic acid, glycolic acid, caprolactone, p-dioxanone (PDO), trimethylene carbonate (TMC), a copolymer, terpolymer, and combinations and mixtures thereof, wherein glycolic acid is the predominant polymer and the polymer has a molecular weight ranging from about 3,000 to about 120,000.

In another aspect, the invention pertains to an implantable elastomeric depot composition for sustained systemic delivery of a beneficial agent to a subject in a controlled manner over a duration equal to or greater than one week after administration, comprising: (a) an elastomeric viscous gel formulation comprising: (1) a bioerodible, biocompatible elastomeric polymer, wherein the polymer is a glycolic acid-based polymer; and (2) a solvent having a miscibility in water of less than or equal to 7 wt. % at 25° C., in an amount effective to plasticize the polymer and form a gel therewith; and (b) a beneficial agent dissolved or dispersed in the gel.

In an additional aspect, the invention pertains to an implantable elastomeric depot composition for sustained delivery of a beneficial agent to a subject in a controlled manner over a predetermined duration of time after administration, comprising (a) a viscous gel formulation comprising: (I) a bioerodible, biocompatible, elastomeric polymer, wherein the polymer is a glycolic acid-based polymer; and (2) a solvent having a miscibility in water of less than or equal to 7 wt. % at 25° C., in an amount effective to plasticize the polymer and form a gel therewith; and (b) a beneficial agent dissolved or dispersed in the gel, wherein the beneficial agent is delivered systemically in a controlled manner over a duration equal to or greater than one week after administration.

In another aspect, the invention pertains to an implantable elastomeric depot composition for sustained local delivery of a beneficial agent to a subject in a controlled manner over a duration equal to or greater than one month after administration, comprising (a) an elastomeric viscous gel formulation comprising: (I) a bioerodible, biocompatible, elastomeric polymer, wherein the polymer is a glycolic acid-based polymer; and (2) a solvent having a miscibility in water of less than or equal to 7 wt. % at 25° C., in an amount effective to plasticize the polymer and form a gel therewith; and (b) a beneficial agent dissolved or dispersed in the gel.

In an additional aspect, the invention pertains to an implantable elastomeric depot composition for sustained delivery of a beneficial agent to a subject in a controlled manner over a predetermined duration of time after administration comprising: (a) an elastomeric viscous gel formulation comprising: (I) a bioerodible, biocompatible, elastomeric polymer, wherein the polymer is a glycolic acid-based polymer; and (2) a solvent having a miscibility in water of less than or equal to 7 wt. % at 25° C., in an amount effective to plasticize the polymer and form a gel therewith; and (b) a beneficial agent dissolved or dispersed in the gel, wherein the beneficial agent is delivered locally in a controlled manner over a duration equal to or greater than one week after administration.

In another aspect, the invention pertains to an implantable elastomeric depot composition as described above, further including at least one of the following: a pore former, a solubility modulator for the beneficial agent, and an osmotic agent.

In another aspect, the invention pertains to an implantable elastomeric depot composition as described above, wherein the elastomeric viscous gel further comprises a polymer selected from the group consisting of polylactides, polyglycolides, poly(caprolactone), polyanhydrides, polyamines, polyesteramides, polyorthoesters, polydioxanones, polyacetals, polyketals, polycarbonates, polyphosphoesters, polyorthocarbonates, polyphosphazenes, succinates, poly(malic acid), poly(amino acids), polyvinylpyrrolidone, polyethylene glycol, polyhydroxycellulose, polyphosphoesters, polysaccharides, chitin, chitosan, hyaluronic acid, p-dioxanone (PDO), trimethylene carbonate (TMC), poly(propylene fumarate), poly(orthoesters), polyphosphoester, and copolymers, terpolymers and mixtures thereof. Additional examples of polymers useful in this invention are described in U.S. Pat. Nos. 6,113,624; 5,868,788; 5,714,551; 5,713,920; 5,639,851 and 5,468,253, which are herein incorporated in their entirety by reference.

In another aspect, the invention pertains to an implantable elastomeric depot composition as described above, wherein the solvent is selected from an aromatic alcohol having the structural formula (I)
Ar-(L)n-OH (I)
in which Ar is a substituted or unsubstituted aryl or heteroaryl group, n is zero or 1, and L is a linking moiety, and a solvent selected from the group consisting of esters of aromatic acids, aromatic ketones, and mixtures thereof.

In preferred embodiments, the solvent is selected from the aromatic alcohol, lower alkyl and aralkyl esters of aryl acids; aryl, aralkyl and lower alkyl ketones; and lower alkyl esters of citric acid. Preferably, the solvent is selected from benzyl alcohol, benzyl benzoate and ethyl benzoate. In preferred embodiments, the composition is free of solvents having a miscibility in water that is greater than 7 wt. % at 25° C. Preferably, the solvent has a miscibility in water of less than 7 wt. %, more preferably less than 5 wt. %, and even more preferably less than 3 wt. %.

In additional aspects, the invention pertains to methods of administering a beneficial agent to a subject in a controlled manner over a duration equal to or greater than one week and up to one year after administration, comprising administering an implantable elastomeric depot composition as described above. In certain embodiments, the beneficial agent is delivered systemically in a controlled manner over a duration equal to or greater than one week and up to one year after administration. In additional embodiments, the beneficial agent is delivered locally in a controlled manner over a duration equal to or greater than one week and up to one year after administration.

In preferred embodiments, the beneficial agent is selected from a drug, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents; anticoagulants, fibrinolytic agents, growth factors, antibodies, ocular drugs, and metabolites, analogs, derivatives, fragments, and purified, isolated, recombinant and chemically synthesized versions of these species. Preferably, the beneficial agent is present in an amount of from 0.1 to 50% by weight of the combined amounts of the polymer, the solvent and the beneficial agent. In preferred embodiments, the beneficial agent is in the form of particles dispersed or dissolved in the viscous gel, wherein the beneficial agent is in the form of particles having an average particle size of from 0.1 to 250 microns. In certain preferred embodiments, the beneficial agent is in the form of particles, wherein the particles further comprise a component selected from the group consisting of a stabilizing agent, bulking agent, chelating agent and a buffering agent.

In additional aspects, the invention pertains to a kit for administration and sustained delivery of a beneficial agent to a subject in a controlled manner over a predetermined duration of time after administration, comprising: (a) a bioerodible, biocompatible, elastomeric polymer, wherein the polymer is a glycolic acid-based polymer; (b) a solvent having a miscibility in water of less than or equal to 7 wt. % at 25° C., in an amount effective to plasticize the polymer and form a gel therewith; (c) a beneficial agent dissolved or dispersed in the gel; and optionally, one or more of the following: (d) an emulsifying agent; (e) a pore former; (f) a solubility modulator for the beneficial agent, optionally associated with the beneficial agent; and (g) an osmotic agent; wherein at least the beneficial agent, optionally associated with the solubility modulator, is maintained separated from the solvent until the time of administration of the beneficial agent to a subject. In additional embodiments, the kit comprises a metering device, such as syringe, catheter, pump, syringe pump, autoinjector and the like.

These and other embodiments of the present invention will readily occur to those of ordinary skill in the art in view of the disclosure herein.

### BRIEF DESCRIPTION OF THE DRAWINGS

The foregoing and other objects, features and advantages of the present invention will be more readily understood upon reading the following detailed description in conjunction with the drawings as described hereinafter.

FIG. 1 is a graph with DSC diagrams illustrating the glass transition temperatures of elastomeric polymers used in the present invention.

FIG. 2 is a graph illustrating the rheological properties of the elastomeric depot compositions of the present invention (formulations 1-5).

FIG. 3 is a graph illustrating the injection forces of the elastomeric depot compositions of the present invention (formulations 1-5).

FIG. 4 is a graph illustrating the rheological properties of the elastomeric depot compositions of the present invention (formulations 6-9).

FIG. 5 is a graph illustrating the injection forces of the elastomeric depot compositions of the present invention as a function of polymer molecular weight.

FIG. 6 is a graph illustrating the rheological properties of the elastomeric depot compositions of the present invention (formulations 10-12).

FIG. 7 is a graph illustrating the injection forces of the elastomeric depot compositions of the present invention as a function of polymer concentration.

FIG. 8 is a graph illustrating the injection forces of the elastomeric depot compositions (formulations 13 and 14) of the present invention as a function of injection speed.

FIG. 9 is a graph illustrating the in vivo release profile of hGH obtained from the elastomeric depot compositions of the present invention (formulations 15 and 16).

### DETAILED DESCRIPTION OF THE INVENTION

The present invention is directed to an implantable elastomeric depot composition for delivery of a beneficial agent to a subject over a prolonged duration of time, wherein the implantable elastomeric depot composition serves as an implanted sustained release beneficial agent delivery system after injection into a patient's body. In particular, the invention provides an implantable elastomeric depot composition with desired elasticity while providing for controlled release of the beneficial agent to the subject being treated, the release being controlled over a period equal to or greater than one week and up to one year after administration, preferably over a period equal to or greater than one month after administration. The present invention also relates to a method of using the implantable elastomeric depot composition to administer a beneficial agent to a patient. The beneficial agent can be administered systemically or locally. In preferred embodiments, the implantable elastomeric depot composition is an injectable elastomeric depot composition. The implantable elastomeric depot composition of the invention has desirable elastic properties making it suitable for delivery of beneficial agents to tight spaces, e.g., tight joint spaces, intradisc spaces, muscles (such as heart tissue), intra-arterial tissue, and the like. Additionally, the implantable elastomeric depot composition provides shear thinning to reduce the injection force significantly, without compromising the release profile of the beneficial agent and maintaining the integrity of the depot gel (i.e., the depot gel remains intact in vivo). In certain embodiments, the implantable elastomeric depot composition provides improved release profiles compared to non-elastomeric formulations, as described in greater detail hereinafter.

The implantable elastomeric depot composition is a gel formed from an elastomeric polymer matrix comprising a bioerodible, biocompatible, elastomeric polymer; a solvent having a miscibility in water of less than or equal to 7 wt. % at 25° C., in an amount effective to plasticize the polymer and form a gel therewith; and a beneficial agent dissolved or dispersed in the gel. The present invention is also directed to a method of systemically or locally administering a beneficial agent to a subject by implanting in the subject an implantable elastomeric depot composition as described above.

By appropriate choice of solvent, water migration from the aqueous environment surrounding the implant system is restricted, and beneficial agent is released to the subject over a period of time, thus providing for delivery of the beneficial agent with a controlled burst of beneficial agent and sustained release thereafter.

It has been found that the release rate and/or duration of release of the beneficial agent from the implantable elastomeric depot composition of the invention can be varied by varying the polymer properties, such as the type of polymer, the molecular weight of the polymer (including the modal distribution of the polymer), and the comonomer ratio of the monomers forming the polymer, the end group of the polymers; the type of solvent; and by varying the polymer/solvent ratios to provide a controlled, sustained release of a beneficial agent over a period equal to or greater than one week and up to one year after administration, preferably over a period equal to or greater than one month after administration. The elastomeric depot composition of the invention provides shear thinning, resulting in significant reduction in the injection force without compromising the release profile of the beneficial agent. The release rate profile and duration can be controlled by the appropriate choice of a polymer (including the ratio of the monomers, e.g., L/G/CL, G/CL, TMC/L/G, CL/PDO, PDO/TMC, PDO/L/G/CL; PDO/L/G/TMC; or PDO/L/G/CL/TMC ratios), the molecular weight of the polymer (LMW, MMW, HMW), the end group of the polymer (acid, ester); a water immiscible solvent, the polymer/solvent ratio, emulsifying agent, pore former, solubility modifier for the beneficial agent, an osmotic agent, and the like.

Additionally, the present invention provides a method of regulating the release of a beneficial agent from an implantable elastomeric depot composition. The duration and the rate of release of the beneficial agent are controlled by the appropriate choice of the biodegradable polymer, the molecular weight of the polymer, the comonomer ratio of the various monomers forming the polymer (e.g., the L/G/CL, G/CL, TMC/L/G, CL/PDO, PDO/TMC, PDO/L/G/CL; PDO/L/G/TMC; or PDO/L/G/CL/TMC ratio for a given polymer), the polymer/solvent ratios, and combinations of these factors, as described in greater detail below. Preferably, the polymer is a lactic acid, glycolic acid, caprolactone, p-dioxanone (PDO), trimethylene carbonate (TMC), a copolymer, terpolymer, and combinations and mixtures thereof, wherein glycolic acid is the predominant polymer. In preferred embodiments, the polymer is a glycolic acid based polymer, e.g., a terpolymer of L/G/CL (wherein glycolide is the predominant component), G/CL and the like.

In some embodiments, pore formers and solubility modulators of the beneficial agent may be added to the implant systems to provide desired release profiles from the implant systems, along with typical pharmaceutical excipients and other additives that do not change the beneficial aspects of the present invention.

The composition provides controlled sustained release of the beneficial agent by restricting water migration from the aqueous environment surrounding the implant system, thus delivering the beneficial agent over a prolonged duration as described earlier. A single administration of the implantable elastomeric depot composition provides longer sustained release of active agents over a prolonged duration of time, thus reducing the frequency of administration and improving patient compliance. Because the polymer of the composition is bioerodible, the implant system does not have to be surgically removed after beneficial agent is depleted from the implant.

Generally, the compositions of the invention are gel-like and form with a substantially homogeneous non-porous structure throughout the implant upon implantation and during drug delivery, even as it hardens. Furthermore, while the polymer gel implant will slowly harden when subjected to an aqueous environment, the hardened implant may maintain a rubbery (non-rigid) composition with the glass transition temperature Tg being below 37° C.

The preferred compositions herein allow beneficial agent to be loaded into the interior of the polymer at levels that are above those required to saturate the beneficial agent in water, thereby facilitating zero order release of beneficial agent. Additionally, the preferred compositions may provide viscous gels that have a glass transition temperature that is less than 37° C., such that the gel remains non-rigid for a period of time after implantation of 24 hours or more.

It has been discovered that when a solvent having a solubility in water of less than 7% by weight in water is present in the system, suitable burst control arid sustained delivery of beneficial agent is achieved, whether or not a solubility modulator of the beneficial agent is present in the system. Typically, the implant systems useful in this invention will release, in the first two days after implantation, 60% or less of the total amount of beneficial agent to be delivered to the subject from the implant system, preferably 50% or less, more preferably 40% or less, more preferably 30% or less, and even more preferably 20% or less.

When the composition is intended for implantation by injection, the viscosity optionally may be modified by addition of emulsifiers or thixotropic agents to obtain a gel composition having a viscosity low enough to permit passage of the gel composition through a needle. Also, pore formers and solubility modulators of the beneficial agent may be added to the implant systems to provide desired release profiles from the implant systems, along with typical pharmaceutical excipients and other additives that do not change the beneficial aspects of the present invention. The addition of a solubility modulator to the implant system may enable the use of a solvent having a solubility of 7% or greater in the implant system with minimal burst and sustained delivery under particular circumstances. However, it is presently preferred that the implant system utilize at least one solvent having a solubility in water of less than 7% by weight, whether the solvent is present alone or as part of a solvent mixture. It has also been discovered that when mixtures of solvents which include a solvent having 7% or less by weight solubility in water and one or more miscible solvents, optionally having greater solubility, are used, implant systems exhibiting limited water uptake and minimal burst and sustained delivery characteristics are obtained.

In describing and claiming the present invention, the following terminology will be used in accordance with the definitions set out below.

The singular forms “a,” “an” and “the” include plural referents unless the context clearly dictates otherwise. Thus, for example, reference to “a solvent” includes a single solvent as well as a mixture of two or more different solvents, reference to “a beneficial agent” includes a single beneficial agent as well as two or more different beneficial agents in combination, and the like.

The term “beneficial agent” means an agent that affects a desired beneficial, often pharmacological, effect upon administration to a human or an animal, whether alone or in combination with other pharmaceutical excipients or inert ingredients.

As used herein, the term “polynucleotide” refers to a polymeric form of nucleotides of any length, either ribonucleotides or deoxyribonucleotides, and includes double- and single-stranded DNA and RNA. It also includes known types of modifications, substitutions, and internucleotide modifications, which are known in the art.

As used herein, the term “recombinant polynucleotide” refers to a polynucleotide of genomic, cDNA, semisynthetic, or synthetic origin which, by virtue of its origin or manipulation: is not associated with all or a portion of a polynucleotide with which it is associated in nature, is linked to a polynucleotide other than that to which it is linked in nature, or does not occur in nature.

As used herein, the term “polypeptide” refers to a polymer of amino acids including, for example, peptides, oligopeptides, and proteins and derivatives, analogs and fragments thereof, as well as other modifications known in the art, both naturally occurring and non-naturally occurring.

As used herein, the terms “purified” and “isolated” when referring to a polypeptide or nucleotide sequence mean that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type present.

The term “AUC” means the area under the curve obtained from an in vivo assay in a subject by plotting blood plasma concentration of the beneficial agent in the subject against time, as measured from the time of implantation of the composition, to a time “t” after implantation. The time t will correspond to the delivery period of beneficial agent to a subject.

The term “burst index” means, with respect to a particular composition intended for systemic delivery of a beneficial agent, the quotient formed by dividing (i) the AUC calculated for the first time period after implantation of the composition into a subject divided by the number of hours in the first time period (t1), by (ii) the AUC calculated for the time period of delivery of the beneficial agent, divided by the number of hours in the total duration of the delivery period (t2). For example, the burst index at 24 hours is the quotient formed by dividing (i) the AUC calculated for the first twenty-four hours after implantation of the composition into a subject divided by the number 24, by (ii) the AUC calculated for the time period of delivery of the beneficial agent, divided by the number of hours in the total duration of the delivery period.

The phrase “dissolved or dispersed” is intended to encompass all means of establishing a presence of beneficial agent in the gel composition and includes dissolution, dispersion, suspension and the like.

The term “systemic” means, with respect to delivery or administration of a beneficial agent to a subject, that the beneficial agent is detectable at a biologically significant level in the blood plasma of the subject.

The term “local” means, with respect to delivery or administration of a beneficial agent to a subject, that the beneficial agent is delivered to a localized site in the subject but is not detectable at a biologically significant level in the blood plasma of the subject.

The terms “prolonged period” or “prolonged duration” are used interchangeably and refer to a period of time over which release of a beneficial agent from the depot composition of the invention occurs, which will generally be over a period equal to or greater than one week and up to one year after administration, preferably over a period equal to or greater than one month after administration, more preferably over a period equal to or greater than two months after administration, even more preferably over a period equal to or greater than three months after administration, preferably within a period of about three months to about nine months after administration, more preferably within a period of about three months to about six months after administration, preferably over a period of up to about six months after administration.

The phrase “gel vehicle” means the composition formed by a mixture of an elastomeric polymer and solvent in the absence of the beneficial agent.

The phrase “initial burst” means, with respect to a particular composition of this invention, the quotient obtained by dividing (i) the amount by weight of beneficial agent released from the composition in a predetermined initial period of time after implantation, by (ii) the total amount of beneficial agent that is to be delivered from an implanted composition. It is understood that the initial burst may vary depending on the shape and surface area of the implant. Accordingly, the percentages and burst indices associated with initial burst described herein are intended to apply to compositions tested in a form resulting from dispensing of the composition from a standard syringe.

The phrase “solubility modulator” means, with respect to the beneficial agent, an agent that will alter the solubility of the beneficial agent, with reference to polymer solvent or water, from the solubility of beneficial agent in the absence of the modulator. The modulator may enhance or retard the solubility of the beneficial agent in the solvent or water. However, in the case of beneficial agents that are highly water soluble, the solubility modulator will generally be an agent that will retard the solubility of the beneficial agent in water. The effects of solubility modulators of the beneficial agent may result from interaction of the solubility modulator with the solvent, or with the beneficial agent itself, such as by the formation of complexes, or with both. For the purposes hereof, when the solubility modulator is “associated” with the beneficial agent, all such interactions or formations as may occur are intended. Solubility modulators may be mixed with the beneficial agent prior to its combination with the viscous gel or may be added to the viscous gel prior to the addition of the beneficial agent, as appropriate.

The terms “subject” and “patient” mean, with respect to the administration of a composition of the invention, an animal or a human being.

The term “thixotropic” is used in its conventional sense to refer to a gel composition that can liquefy or at least exhibit a decrease in apparent viscosity upon application of mechanical force such as shear force. The extent of the reduction is in part a function of the shear rate of the gel when subjected to the shearing force. When the shearing force is removed, the viscosity of the thixotropic gel returns to a viscosity at or near that which it displayed prior to being subjected to the shearing force. Accordingly, a thixotropic gel may be subjected to a shearing force when injected from a syringe which temporarily reduces its viscosity during the injection process. When the injection process is completed, the shearing force is removed and the gel returns very near to its previous state.

A “thixotropic agent” as used herein is one that increases the thixotropy of the composition in which it is contained, promoting shear thinning and enabling use of reduced injection force.

The term “bioerodible” refers to a material that gradually decomposes, dissolves, hydrolyzes and/or erodes in situ. Generally, the “bioerodible” polymers-herein are polymers that are hydrolyzable, and bioerode in situ primarily through hydrolysis.

The terms “elastomer” or “elastomeric polymer” refer to a material having a subambient glass transition temperature, and elongation properties.

The phrase “low molecular weight (LMW) polymer” refers to bioerodible polymers having a weight average molecular weight ranging from about 3000 to about 10,000, preferably from about 3000 to about 9,000, more preferably from about 4000 to about 8,000, and more preferably the low molecular weight polymer has a molecular weight of about 7000, about 6000, about 5000, about 4000 and about 3000 as determined by gel permeation chromatography (GPC).

The phrase “medium molecular weight (MMW) polymer” refers to biocompatible, bioerodible polymers having a weight average molecular weight ranging from between about 10,000 to about 30,000, preferably from about 12,000 to about 20,000, more preferably from about 14,000 to about 18,000, and more preferably the medium molecular weight polymer has a molecular weight of about 14,000, about 15,000, about 16,000, about 17,000 and about 18,000 as determined by gel permeation chromatography (GPC). The phrase “high molecular weight (HMW) polymer” refers to biocompatible, bioerodible polymers having a weight average molecular weight of greater than 30,000, preferably from about 30,000 to about 250,000, more preferably from about 30,000 to about 120,000 as determined by gel permeation chromatography (GPC).

Since all solvents, at least on a molecular level, will be soluble in water (i.e., miscible with water) to some very limited extent, the term “immiscible” as used herein means that 7% or less by weight, preferably 5% or less, of the solvent is soluble in or miscible with water. For the purposes of this disclosure, solubility values of solvent in water are considered to be determined at 25° C. Since it is generally recognized that solubility values as reported may not always be conducted at the same conditions, solubility limits recited herein as percent by weight miscible or soluble with water as part of a range or upper limit may not be absolute. For example, if the upper limit on solvent solubility in water is recited herein as “7% by weight,” and no further limitations on the solvent are provided, the solvent “triacetin,” which has a reported solubility in water of 7.17 grams in 100 ml of water, is considered to be included within the limit of 7%. A solubility limit in water of less than 7% by weight as used herein does not include the solvent triacetin or solvents having solubilities in water equal to or greater than triacetin.

The following definitions apply to the molecular structures described herein. As used herein, the phrases “having the formula” or “having the structure” are not intended to be limiting and are used in the same way that the term “comprising” is commonly used.

The term “alkyl” as used herein refers to a saturated hydrocarbon group typically, although not necessarily, containing 1 to about 30 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The phrase “lower alkyl” means an alkyl group of 1 to 6 carbon atoms, and more preferably 1 to 4 carbon atoms. “Substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl or lower alkyl.

The term “aryl” as used herein, and unless otherwise specified, refers to an aromatic substituent containing a single aromatic ring or multiple aromatic rings that are fused together, linked covalently, or linked to a common group such as a methylene or ethylene moiety. Preferred aryl groups contain one aromatic ring or two fused or linked aromatic rings, e.g., phenyl, naphthyl, biphenyl, diphenylether, diphenylamine, benzophenone, and the like, and most preferred aryl groups are monocyclic. “Substituted aryl” refers to an aryl moiety substituted with one or more substituent groups, and the terms “heteroatom-containing aryl” and “heteroaryl” refer to aryl in which at least one carbon atom is replaced with a heteroatom. Unless otherwise indicated, the term “aryl” includes heteroaryl, substituted aryl, and substituted heteroaryl groups.

The term “aralkyl” refers to an alkyl group substituted with an aryl group, wherein alkyl and aryl are as defined above. The term “heteroaralkyl” refers to an alkyl group substituted with a heteroaryl group. Unless otherwise indicated, the term “aralkyl” includes heteroaralkyl and substituted aralkyl groups as well as unsubstituted aralkyl groups. Generally, the term “aralkyl” herein refers to an aryl-substituted lower alkyl group, preferably a phenyl substituted lower alkyl group such as benzyl, phenethyl, 1-phenylpropyl, 2-phenylpropyl, and the like.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon. Similarly, the term “heterocyclic” refers to a cyclic substituent that is heteroatom-containing, the term “heteroaryl” refers to an aryl substituent that is heteroatom-containing, and the like.

By “substituted” as in “substituted alkyl,” “substituted aryl” and the like, as alluded to in some of the aforementioned definitions, it is meant that in the alkyl or aryl moiety, respectively, at least one hydrogen atom bound to a carbon atom is replaced with one or more non-interfering substituents such as hydroxyl, alkoxy, thio, amino, halo, and the like.

1. Implantable Elastomeric Depot Compositions:

As previously described, implantable elastomeric depot compositions for delivery of beneficial agents over a prolonged period of time may be formed as viscous gels prior to injection of the depot into a subject. The viscous gel supports dispersed beneficial agent to provide appropriate delivery profiles, which include those having low initial burst, of the beneficial agent as the beneficial agent is released from the depot over time.

The polymer, solvent and other agents of the invention must be biocompatible, that is they must not cause irritation or necrosis in the environment of use. The environment of use is a fluid environment and may comprise a subcutaneous, intramuscular, intravascular (high/low flow), intramyocardial, adventitial, intratumoral, or intracerebral portion, wound sites, tight joint spaces or body cavity of a human or animal. In certain embodiments, the beneficial agent may be administered locally to avoid or minimize systemic side effects. Gels of the present invention containing a beneficial agent may be injected/implanted directly into or applied as a coating to the desired location (e.g., subcutaneous, intramuscular, intravascular, intramyocardial, adventitial, intratumoral, or intracerebral portion), wound sites, tight joint spaces or body cavity of a human or animal (e.g., tight joint spaces, intradisc spaces), muscles (such as heart tissue), intra-arterial tissue, and the like.

Typically, the viscous gel will be injected from a standard hypodermic syringe through a needle, a catheter, or a trocar, that has been pre-filled with the beneficial agent-viscous gel composition to form the depot. It is often preferred that injections take place using the smallest size needle (i.e., smallest diameter) to reduce discomfort to the subject when the injection is in a subcutaneous, intramuscular, intravascular (high/low flow), intramyocardial, adventitial, intratumoral, or intracerebral portion, wound sites, tight joint spaces or body cavity of a human or animal. It is desirable to be able to inject gels through a needle or a catheter ranging from 16 gauge and higher, preferably 20 gauge and higher, more preferably 22 gauge and higher, even more preferably 24 gauge and higher. With highly viscous gels, i.e., gels having a viscosity of about 200 poise or greater, injection forces to dispense the gel from a syringe having a needle in the 20 to 30 gauge range may be so high as to make the injection difficult or reasonably impossible when done manually. At the same time, the high viscosity of the gel is desirable to maintain the integrity of the depot after injection and during the dispensing period and also to facilitate desired suspension characteristics of the beneficial agent in the gel.

A. The Bioerodible, Biocompatible, Elastomeric Polymer:

Polymers that are useful in conjunction with the methods and compositions of the invention are bioerodible, i.e., they gradually degrade, e.g., enzymatically or hydrolyze, dissolve, physically erode, or otherwise disintegrate within the aqueous fluids of a patient's body. Generally, the polymers bioerode as a result of hydrolysis or physical erosion, although the primary bioerosion process is typically hydrolysis or enzymatic degradation. Additionally, the polymers that are useful in this invention when formulated in a gel are elastomeric and exhibit a desirable degree of elasticity while retaining the integrity of the gel and providing a desirable release profile for the beneficial agent.

Such polymers include, but are not limited to, polylactides, polyglycolides, polycaprolactones, polyanhydrides, polyamines, polyesteramides, polyorthoesters, polydioxanones, polyacetals, polyketals, polycarbonates, polyorthocarbonates, polyphosphazenes, succinates, poly(malic acid), poly(amino acids), polyvinylpyrrolidone, polyethylene glycol, polyhydroxycellulose, hydroxymethylcellulose polyphosphoesters, polysaccharides, chitin, chitosan, hyaluronic acid and copolymers, terpolymers and mixtures thereof. Additional examples of polymers useful in this invention are described in U.S. Pat. Nos. 6,113,624; 5,868,788; 5,714,551; 5,713,920; 5,639,851 and 5,468,253, which are herein incorporated in their entirety by reference.

It has been found that the release rate and/or duration of release of the beneficial agent from the implantable elastomeric depot compositions of the invention can be varied by varying the polymer properties, such as the type of polymer, the molecular weight of the polymer (including the modal distribution of the polymer), and the comonomer ratio of the monomers forming the polymer; the end group of the polymers; the type of solvent; and by varying the polymer/solvent ratios to provide a controlled, sustained release of a beneficial agent over a period equal to or greater than one week and up to one year after administration, preferably over a period equal to or greater than one month after administration. The release rate profile and duration can be controlled by the appropriate choice of a polymer (including the ratio of the monomers, e.g. L/G/CL or G/CL ratios), the molecular weight of the polymer (LMW, MMW, HMW), the end group of the polymer (acid, ester); a water immiscible solvent, the polymer/solvent ratio, emulsifying agent, pore former, solubility modifier for the beneficial agent, as osmotic agent, and the like.

In another aspect, the present invention provides a method of regulating the release of a beneficial agent from an implantable elastomeric depot composition. The duration and the rate of release of the beneficial agent (e.g., burst index and release rate profile) are controlled by the appropriate choice of the biodegradable polymer, the molecular weight of the polymer, the comonomer ratio of the various monomers forming the polymer (e.g., the L/G/CL or G/CL ratio for a glycolic acid-based polymer), and the polymer/solvent ratios. Previously described injectable depot formulations having predominantly polylactic acid components are not bioabsorbable. As illustrated in the Examples below, it has been discovered that elastomeric depot compositions of the invention, preferably compositions wherein glycolic acid is the predominant component, have desirable elastomeric properties without compromising the release profiles of the beneficial agent.

In one aspect, duration and the rate of release (e.g., release rate profile and burst index) of the beneficial agent are controlled by the appropriate choice of the biodegradable polymer.

Molecular weight of the polymer: The molecular weight of the polymer can be varied to regulate the release rate profile and/or delivery duration of the beneficial agent. In general, as the molecular weight of the polymer increases, one or more of the following occurs: the burst index is lower, release rate profile is flatter and/or duration of delivery is longer.

Polymers with different end groups: Implantable elastomeric depot compositions having a blend of polymers with different end groups would result in a depot formulation having a lower burst index and a regulated duration of delivery. For example, blending PLGA RG502H (acid end group) with PLGA RG502 (ester end group) lowers the burst index for a depot composition having a one month duration of delivery; blending PLGA RG752H with PLGA RG752 lowers the burst index for a depot composition having a duration of delivery of about three months to about four months; blending PLA R202H with PLA R202 lowers the burst index for a depot composition having duration of delivery greater than or equal to six months; blending PLGA RG502H and PLGA RG752 with PLA R202 lowers the burst index for a depot composition having a duration of delivery up to six months.

Comonomer ratio of the polymer: Varying the comonomer ratio of the various monomers forming the polymer (e.g., the L/G/CL or G/CL ratio for a given polymer), would result in depot compositions having a regulated burst index and duration of delivery. For example, a depot composition having a polymer with a L/G ratio of 50:50 has a short duration of delivery ranging from two days to about one month; a depot composition having a polymer with a L/G ratio of 65:35 has a duration of delivery of about two months; a depot composition having a polymer with a L/G ratio of 75:25 or L/CL ratio of 75:25 has a duration of delivery of about three months to about four months; a depot composition having a polymer with a L/G ratio of 85:15 has a duration of delivery of about five months; a depot composition having a polymer with a L/CL ratio of 25:75 or PLA has a duration of delivery greater than or equal to six months; a depot composition-having a terpolymer of CL/G/L with G greater than 50% and L greater than 10% has a duration of delivery about one month and a depot composition having a terpolymer of CL/G/L with G less than 50% and L less than 10% has a duration of delivery of about two months up to six months.

Polymers with different degradation characteristics: Depot compositions having a blend of a faster degrading polymer with a slower degrading polymer would result in a depot formulation having a lower burst index and a flatter release rate profile. For example, blending PLGA RG502 with PLGA RG752 would yield a depot composition having a lower burst index (as compared to a gel composition having PLGA RG752 alone) and a duration of delivery of about three months to about four months after administration. Blending PLGA RG502 and PLGA RG752 with PLA R202 would yield a depot composition having a lower burst index (as compared to a gel composition having PLA 202 alone) and a duration of delivery greater than or equal to six months after administration.

Polymers with different molecular weights, end group and comonomer ratios: Depot compositions having a blend of polymers having different molecular weights, end group and comonomer ratios result in a depot formulation having a lower burst index and a regulated duration of delivery. For example, blending LMW PLGA (L/G: 50/50) and PLGA RG502H (acid end group) with PLGA RG502 (ester end group) would yield a depot composition having a lower burst index (as compared to a gel composition having PLGA RG502 alone) and a duration of delivery of about one month. Blending LMW PLGA (L/G: 50/50) and PLGA RG503H (acid end group) with PLGA RG752 (ester end group) would yield a depot composition having a lower burst index (as compared to a gel composition having PLGA RG752 alone) and a duration of delivery of about three months to about four months after administration. Blending LMW PLGA (L/G: 50/50) and PLGA RG755H (acid end group) with PLA R202 (ester end group) would yield a depot composition having a lower burst index (as compared to a gel composition having PLA 202 alone) and a duration of delivery greater than or equal to six months after administration. Blending PLGA RG502H (acid end group) and PLGA RG752 (ester end group) with PLA R206 (ester end group) would yield a depot composition having a lower burst index (as compared to a gel composition having PLA 202 alone) and a duration of delivery greater than or equal to six months after administration.

In another aspect, duration and the rate of release of the beneficial agent are controlled by varying the polymer/solvent (P/S) ratio. The polymer/solvent ratio of the depot composition can be varied to regulate the release rate profile and/or delivery duration of the beneficial agent. In general, the higher the P/S ratio, the lower the burst index or flatter release rate profile.

The bioerodible polymers are selected from the group consisting of low molecular weight (LMW) polymers, medium molecular weight (MMW) polymers and high molecular weight (HMW) polymers. The low molecular weight (LMW) bioerodible polymers have weight average molecular weight ranging from about 3000 to about 10,000, preferably from about 3000 to about 9,000, more preferably from about 4000 to about 8,000, and most preferably the low molecular weight polymer has a molecular weight of about 7000, about 6000, about 5000, about 4000 and about 3000 as determined by gel permeation chromatography (GPC).

The medium molecular weight (MMW) bioerodible polymers have weight average molecular weights ranging from between about 10,000 to about 30,000, preferably from about 12,000 to about 20,000, more preferably from about 14,000 to about 18,000, and most preferably the medium molecular weight polymer has a molecular weight of about 14,000, about 15,000, about 16,000, about 17,000 and about 18,000 as determined by gel permeation chromatography (GPC).

The high molecular weight (HMW) bioerodible polymers have weight average molecular weights of greater than 30,000, preferably from about 30,000 to about 250,000, more preferably from about 30,000 to about 120,000 as determined by gel permeation chromatography (GPC).

Preferably, the polymer matrix comprises about 0 wt. % to about 95 wt. % of low molecular weight (LMW) polymer, preferably about 20 wt. % to about 90 wt. % of low molecular weight (LMW) polymer, more preferably about 30 wt. % to about 80 wt. % of low molecular weight (LMW) polymer, and more preferably about 40 wt. % to about 75 wt. % of low molecular weight (LMW) polymer; about 0 wt. % to about 50 wt. % of high molecular weight (HMW) polymer, preferably about 5 wt. % to about 40 wt. % of high molecular weight (HMW) polymer, more preferably about 10 wt. % to about 30 wt. % of high molecular weight (HMW) polymer, and more preferably about 15 wt. % to about 25 wt. % of high molecular weight (HMW) polymer; and about 0 wt. % to about 95 wt. % of medium molecular weight (MMW) polymer, preferably about 20 wt. % to about 90 wt. % of medium molecular weight (MMW) polymer, more preferably about 30 wt. % to about 80 wt. % of medium molecular weight (MMW) polymer, and more preferably about 40 wt. % to about 65 wt. % of medium molecular weight (MMW) polymer.

Preferably the polymer is a lactic acid, glycolic acid, caprolactone, p-dioxanone (PDO), trimethylene carbonate (TMC), a copolymer, terpolymer, and combinations and mixtures thereof, wherein glycolic acid is the predominant polymer. Presently preferred polymers are polyglycolides, that is, a glycolic acid-based polymer that can be based solely on glycolic acid or can be a copolymer or a terpolymer based on lactic acid, glycolic acid, caprolactone (CL), trimethylene carbonate (TMC) and/or p-dioxanone (PDO) wherein the glycolic acid is the predominant component, and which may include small amounts of other comonomers that do not substantially affect the advantageous results, which can be achieved in accordance with the present invention. In preferred embodiments, the polymer is a glycolic acid based polymer, e.g., a terpolymer of L/G/CL wherein glycolide is the predominant component, G/CL and the like. As used herein, the term “lactic acid” includes the isomers L-lactic acid, D-lactic acid, DL-lactic acid and lactide while the term “glycolic acid” includes glycolide. Most preferred are polymers selected from the group consisting of polylactide polymers, commonly referred to as PLA, poly(lactide-co-glycolide) copolymers, commonly referred to as PLGA, and poly(caprolactone-co-lactic acid) (PCL-co-LA). The polymer may have a monomer ratio of lactic acid/glycolic acid (L/G) of from about 50:50 to about 100:0, preferably from about 60:40 to about 85:15, preferably from about 65:35 to about 75:25. In certain embodiments, when the desired duration of release of the beneficial agent is about one month, preferably the polymer has a L/G ratio of 50:50. In alternative embodiments, when the desired duration of release of the beneficial agent is about two months, preferably the polymer has a L/G ratio of 65:35; when the desired duration of release of the beneficial agent is about three months, preferably the polymer has a L/G ratio of 75:25; and when the desired duration of release of the beneficial agent is about six months, preferably the polymer has a L/G ratio ranging from about 85:15 to about 100:0.

The poly(caprolactone-co-lactic acid) (PCL-co-LA) polymer has a comonomer ratio of caprolactone/lactic acid (CL/L) of from about 10:90 to about 90:10, from about 50:50, preferably from about 35:65 to about 65:35, and more preferably from about 25:75 to about 75:25. In certain embodiments, the lactic acid based polymer comprises a blend of about 0-90% caprolactone, about 0-100% lactic acid, and about 0-60% glycolic acid.

As indicated in aforementioned U.S. Pat. No. 5,242,910, the polymer can be prepared in accordance with the teachings of U.S. Pat. No. 4,443,340. Alternatively, the glycolic acid-based polymer can be prepared directly from lactic acid or a mixture of lactic acid, glycolic acid and or caprolactone (with or without a further comonomer) in accordance with the techniques set forth in U.S. Pat. No. 5,310,865. The contents of all of these patents are incorporated by reference. Suitable glycolic and lactic acid-based polymers are available commercially. The glycolic acid-based polymer may be a low molecular weight polymer (LMW), a medium molecular weight polymer (MMW) or a high molecular weight (HMW) or a combination thereof.

Examples of polymers include, but are not limited to, Poly(D,L-lactide-co-glycolide) 50:50 Resomer® RG502, Poly(D,L-lactide-co-glycolide) 50:50 Resomer® RG502H, Poly D,L Lactide (Resomer® R 202, Resomer® R 203); Poly dioxanone (Resomer® X 210) (Boehringer Ingelheim Chemicals, Inc., Petersburg, Va.). Additional examples include, but are not limited to, DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinnati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly F-caprolactone; Poly DL-lactide-co-caprolactone 25:75; and Poly DL-lactide-co-caprolactone 75:25 (Birmingham Polymers, Inc., Birmingham, Ala.). Additional examples of polymers useful in this invention are described in U.S. Pat. Nos. 6,113,624; 5,868,788; 5,714,551; 5,713,920; 5,639,851 and 5,468,253.

The biocompatible polymer is present in the gel composition in an amount ranging from about 5 to about 90% by weight, preferably from about 10 to about 80% by weight, preferably from about 20 to about 75% by weight, often about 30 to about 70% by weight of the viscous gel, and about 35 to about 65% by weight of the viscous gel comprising the combined amounts of the biocompatible polymer and the solvent. The solvent will be added to polymer in the amounts described below, to provide implantable elastomeric depot compositions.

B. Solvents:

The implantable elastomeric depot composition of the invention contains a water-immiscible solvent in addition to the bioerodible polymer and the beneficial agent. In preferred embodiments, the compositions described herein are also free of solvents having a miscibility in water that is greater than 7 wt. % at 25° C.

The solvent must be biocompatible, should form a viscous gel with the polymer, and restrict water uptake into the implant. The solvent may be a single solvent or a mixture of solvents exhibiting the foregoing properties. The term “solvent,” unless specifically indicated otherwise, means a single solvent or a mixture of solvents. Suitable solvents will substantially restrict the uptake of water by the implant and may be characterized as immiscible in water, i.e., having a solubility in water of less than 7% by weight. Preferably, the solvents are 5 wt. % or less soluble in water, more preferably 3 wt. % or less soluble in water, and even more preferably 1 wt. % or less soluble in water. Most preferably, the solubility of the solvent in water is equal to or less than 0.5 wt. %.

Water miscibility may be determined experimentally as follows: Water (1-5 g) is placed in a tared clear container at a controlled temperature, about 20° C., and weighed, and a candidate solvent is added dropwise. The solution is swirled to observe phase separation. When the saturation point appears to be reached, as determined by observation of phase separation, the solution is allowed to stand overnight and is rechecked the following day. If the solution is still saturated, as determined by observation of phase separation, then the percent (w/w) of solvent added is determined. Otherwise more solvent is added and the process is repeated. Solubility or miscibility is determined by dividing the total weight of solvent added by the final weight of the solvent/water mixture. When solvent mixtures are used, for example 20% triacetin and 80% benzyl benzoate, they are premixed prior to adding to the water.

Solvents useful in this invention are generally less than 7% water soluble by weight as described above. Solvents having the above solubility parameter may be selected from aromatic alcohols, the lower alkyl and aralkyl esters of aryl acids such as benzoic acid, the phthalic acids, salicylic acid, lower alkyl esters of citric acid, such as triethyl citrate and tributyl citrate and the like, and aryl, aralkyl and lower alkyl ketones. Among preferred solvents are those having solubilities within the foregoing range selected from compounds having the following structural formulas (I), (II) and (III).

The aromatic alcohol has the structural formula (I)
Ar-(L)n-OH (I)
wherein Ar is a substituted or unsubstituted aryl or heteroaryl group, n is zero or 1, and L is a linking moiety. Preferably, Ar is a monocyclic aryl or heteroaryl group, optionally substituted with one or more noninterfering substituents such as hydroxyl, alkoxy, thio, amino, halo, and the like. More preferably, Ar is an unsubstituted 5- or 6-membered aryl or heteroaryl group such as phenyl, cyclopentadienyl, pyridinyl, pyrimadinyl, pyrazinyl, pyrrolyl, pyrazolyl, imidazolyl, furanyl, thiophenyl, thiazolyl, isothiazolyl, or the like. The subscript “n” is zero or 1, meaning that the linking moiety L may or may not be present. Preferably, n is 1 and L is generally a lower alkylene linkage such as methylene or ethylene, wherein the linkage may include heteroatoms such as O, N or S. Most preferably, Ar is phenyl, n is 1, and L is methylene, such that the aromatic alcohol is benzyl alcohol.

The aromatic acid ester or ketone may be selected from the lower alkyl and aralkyl esters of aromatic acids, and aryl and aralkyl ketones. Generally, although not necessarily, the aromatic acid esters and ketones will respectively have the structural formula (II) or (III):

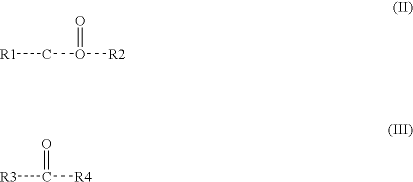

In the ester of formula (II), R1 is substituted or unsubstituted aryl, aralkyl, heteroaryl or heteroaralkyl, preferably substituted or unsubstituted aryl or heteroaryl, more preferably monocyclic or bicyclic aryl or heteroaryl optionally substituted with one or more non-interfering substituents such as hydroxyl, carboxyl, alkoxy, thio, amino, halo, and the like, still more preferably 5- or 6-membered aryl or heteroaryl such as phenyl, cyclopentadienyl, pyridinyl, pyrimadinyl, pyrazinyl, pyrrolyl, pyrazolyl, imidazolyl, furanyl, thiophenyl, thiazolyl, or isothiazolyl, and most preferably 5- or 6-membered aryl. R2 is hydrocarbyl or heteroatom-substituted hydrocarbyl, typically lower alkyl or substituted or unsubstituted aryl, aralkyl, heteroaryl or heteroaralkyl, preferably lower alkyl or substituted or unsubstituted aralkyl or heteroaralkyl, more preferably lower alkyl or monocyclic or bicyclic aralkyl or heteroaralkyl optionally substituted with one or more non-interfering substituents such as hydroxyl, carboxyl, alkoxy, thio, amino, halo, and the like, still more preferably lower alkyl or 5- or 6-membered aralkyl or heteroaralkyl, and most preferably lower alkyl or 5- or 6-membered aryl optionally substituted with one or more additional ester groups having the structure —O—(CO)—R1. Most preferred esters are benzoic acid and phthalic acid derivatives.

In the ketone of formula (III), R3 and R4 may be selected from any of the R1 and R2 groups identified above.

Art recognized benzoic acid derivatives from which solvents having the requisite solubility may be selected include, without limitation: 1,4-cyclohexane dimethanol dibenzoate, diethylene glycol dibenzoate, dipropylene glycol dibenzoate, polypropylene glycol dibenzoate, propylene glycol dibenzoate, diethylene glycol benzoate and dipropylene glycol benzoate blend, polyethylene glycol (200) dibenzoate, isodecyl benzoate, neopentyl glycol dibenzoate, glyceryl tribenzoate, pentaerythritol tetrabenzoate, cumylphenyl benzoate, trimethyl pentanediol dibenzoate.

Art recognized phthalic acid derivatives from which solvents having the requisite solubility may be selected include: Alkyl benzyl phthalate, bis-cumyl-phenyl isophthalate, dibutoxyethyl phthalate, dimethyl phthalate, dimethyl phthalate, diethyl phthalate, dibutyl phthalate, diisobutyl phthalate, butyl octyl phthalate, diisoheptyl phthalate, butyl octyl phthalate, diisononyl phthalate, nonyl undecyl phthalate, dioctyl phthalate, di-isooctyl phthalate, dicapryl phthalate, mixed alcohol phthalate, di-(2-ethylhexyl) phthalate, linear heptyl, nonyl, phthalate, linear heptyl, nonyl, undecyl phthalate, linear nonyl phthalate, linear nonyl undecyl phthalate, linear dinonyl, didecyl phthalate (di-isodecyl phthalate), diundecyl phthalate, ditridecyl phthalate, undecyldodecyl phthalate, decyltridecyl phthalate, blend (50/50) of dioctyl and didecyl phthalates, butyl benzyl phthalate, and dicyclohexyl phthalate.

Many of the solvents useful in the invention are available commercially (Aldrich Chemicals, Sigma Chemicals) or may be prepared by conventional esterification of the respective arylalkanoic acids using acid halides, and optionally esterification catalysts, such as described in U.S. Pat. No. 5,556,905, which is incorporated herein by reference, and in the case of ketones, oxidation of their respective secondary alcohol precursors.

Preferred solvents include aromatic alcohols, the lower alkyl and aralkyl esters of the aryl acids described above. Representative acids are benzoic acid and the phthalic acids, such as phthalic acid, isophthalic acid, and terephthalic acid. Most preferred solvents are benzyl alcohol and derivatives of benzoic acid and include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate, with benzyl benzoate being most especially preferred.

The composition may also include, in addition to the water-immiscible solvent(s), one or more additional miscible solvents (“component solvents”), provided that any such additional solvent is other than a lower alkanol. Component solvents compatible and miscible with the primary solvent(s) may have a higher miscibility with water and the resulting mixtures may still exhibit significant restriction of water uptake into the implant. Such mixtures will be referred to as “component solvent mixtures.” Useful component solvent mixtures may exhibit solubilities in water greater than the primary solvents themselves, typically between 0.1 wt. % and up to and including 50 wt. %, preferably up to and including 30 wt. %, and most preferably up to and including 10 wt. %, without detrimentally affecting the restriction of water uptake exhibited by the implants of the invention.

Component solvents useful in component solvent mixtures are those solvents that are miscible with the primary solvent or solvent mixture, and include, but are not limited, to triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glycerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, glycofurol, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and mixtures thereof.

Preferred solvent mixtures are those in which benzyl benzoate is the primary solvent, and mixtures formed of benzyl benzoate and either triacetin, tributyl citrate, triethyl citrate or N-methyl-2-pyrrolidone, or glycofurol. Preferred mixtures are those in which benzyl benzoate is present by weight in an amount of 50% or more, more preferably 60% or more and most preferably 80% or more of the total amount of solvent present. Especially preferred mixtures are those of 80:20 mixtures by weight of benzyl benzoate/triacetin and benzyl benzoate/N-methyl-2-pyrrolidone. In additional embodiments, the preferred solvent is benzyl alcohol, and mixtures formed of benzyl alcohol and either benzyl benzoate or ethyl benzoate. Preferred mixtures of benzyl alcohol/benzyl benzoate and benzyl alcohol/ethyl benzoate are 1/99 mixtures by weight, 20/80 mixtures by weight, 30/70 mixtures by weight, 50/50 mixtures by weight, 70/30 mixtures by weight, 80/20 mixtures by weight, 99/1 mixtures by weight. Especially preferred mixtures of benzyl alcohol/benzyl benzoate and benzyl alcohol/ethyl benzoate are 25/75 mixtures by weight and 75/25 mixtures by weight.

The solvent or solvent mixture is typically present in an amount of from about 95 to about 10% by weight, preferably from about 80 to about 20% by weight, preferably about 70-25% by weight, preferably about 65-30% by weight and often 60-40% by weight of the viscous gel, i.e., the combined amounts of the polymer and the solvent. The polymer to solvent ratio ranges from about 20:80 to about 90:10 by weight, preferably about 30:70 to about 80:20 by weight, preferably about 40:60 to about 75:25 by weight, and more preferably about 45:55 to about 65:35 by weight.

In an especially preferred embodiment, the primary solvent is selected from an aromatic alcohol and lower alkyl and aralkyl esters of benzoic acid and the polymer is a lactic-acid based polymer, most preferably selected from polylactide polymers (PLA), poly(lactide-co-glycolide) copolymers (PLGA), and poly(caprolactone-co-lactic acid) (PCL-co-LA) having a comonomer L/G ratio of about 50:50 to about 100:0 and an L/CL ratio of about 25:75 to about 75:25, and a polymer solvent ratio of about 40:60 to about 65:35. Preferably, the polymer has a weight average molecular weight ranging from about 3,000 to about 120,000, preferably from about 7,000 to about 1-00,000, more preferably from about 10,000 to about 80,000, and more preferably the polymer has a molecular weight of about 14,000, about 16,000, about 20,000, about 30,000 and about 60,000.

Presently, the most preferred solvents are benzyl alcohol, benzyl benzoate and the lower alkyl esters of benzoic acid, e.g., ethyl benzoate. The primary solvents, e.g., aromatic alcohol and benzoic acid esters may be used alone or in a mixture with other miscible solvents, e.g., triacetin, or thixotropic agents, e.g., ethanol, as described herein.

The solvent or solvent mixture is capable of dissolving the polymer to form a viscous gel that can maintain particles of the beneficial agent dissolved or dispersed and isolated from the environment of use prior to release. The compositions of the present invention provide implants useful both for systemic and local administration of beneficial agent, the implants having a low burst index. Water uptake is controlled by the use of a solvent or component solvent mixture that solubilizes or plasticizes the polymer but substantially restricts uptake of water into the implant. Additionally, the preferred compositions may provide viscous gels that have a glass transition temperature that is less than 37° C., such that the gel remains non-rigid for a period of time after implantation of 24 hours or more.

The importance of restriction of water uptake and the appropriate choice of a polymer and a water immiscible solvent for a controlled, sustained delivery over a short duration can be appreciated by reference to in vivo release rate profiles for various compositions as a function of time.

In addition to the control of water uptake and associated initial burst by choice of solvent, agents that modulate the water solubility of the beneficial agent can also be utilized in conjunction with the preferred solvents to control burst of beneficial agent from the implant. Burst indices and percent of beneficial agent released in the first twenty-four hours after implantation may be reduced by one-third to two-thirds or more by the use of solubility modulators associated with the beneficial agent. Such modulators are typically coatings, substances that form complexes or otherwise associate with or stabilize the beneficial agent, such as metallic ions, other stabilizing agents, waxes, lipids, oils, non-polar emulsions, and the like. Use of such solubility modulators may permit the use of more highly water soluble solvents or mixtures and achieve burst indices of eight or less for systemic applications, or with respect to local applications. Typically, the implant systems useful in this invention will release, in the first two days after implantation, 60% or less of the total amount of beneficial agent to be delivered to the subject from the implant system, preferably 50% or less, more preferably 40% or less and even more preferably 30% or less.

Limited water uptake by the compositions of this invention can often provide the opportunity to prepare compositions without solubility modulators when in other compositions such modulators would be necessary.

In instances where the choice of solvent and polymer result in compositions severely restricting water uptake by themselves, it may be desirable to add osmotic agents or other agents and hydroattractants that facilitate water uptake to desired levels. Such agents may be, for example, sugars and the like, and are well known in the art.

Limited water uptake by the solvent-polymer compositions of the present invention results in the implant compositions being formed without the finger-like pores in the surface of implants formed using prior art processes. Typically, a composition of the present invention takes the form of a substantially homogeneous, sponge-like gel, with the pores in the interior of the implant being much the same as the pores on the surface of the implant. Compositions of the present invention retain their gel-like consistency and administer a beneficial agent in a controlled manner, at a sustained rate over a short duration of time than do prior art devices. This is possible with the appropriate choice of polymers and water immiscible solvents, and further since the implantable elastomeric depot compositions of the present invention generally have a glass transition temperature, Tg, of less than body temperature of the subject, e.g., 37° C. for humans. Because of the immiscibility of the solvents that are useful in this invention with water, water uptake by the implant is restricted and the pores that do form tend to resemble a closed cell structure without significant numbers of larger pores or pores extending from the surface into the interior of the implant being open at the surface of the implant. Furthermore, the surface pores offer only a limited opportunity for water from body fluids to enter the implant immediately after implantation, thus controlling the burst effect. Since the compositions often will be highly viscous prior to implantation, when the composition is intended for implantation by injection, the viscosity optionally may be modified by the use of viscosity-reducing, miscible solvents or the use of emulsifiers, or by heating to obtain a gel composition having a viscosity or shear resistance low enough to permit passage of the gel composition through a needle.

The limit on the amount of beneficial agent released in the first 24 hours that is either desired or required will depend on circumstances such as the overall duration of the delivery period, the therapeutic window for the beneficial agent, potential adverse consequences due to overdosing, the cost of beneficial agent, and the type of effect desired, e.g., systemic or local. Preferably, 60% or less of the beneficial agent will be released in the first two days after implantation, preferably 50% or less, more preferably 40% or less and even more preferably 30% or less, where the percentage is based on the total amount of beneficial agent to be delivered over the duration of the delivery period.

Depending on the particular solvent or solvent mixture selected, the polymer and beneficial agent, and optionally solubility modulators of the beneficial agent, the compositions of the present invention intended for systemic delivery may provide a gel composition having a burst index of eight or less, preferably six or less, more preferably four or less and most preferably two or less. Compositions of the elastomeric polymers weight average molecular weight ranging from about 3,000 to about 120,000, preferably from about 7,000 to about 100,000, more preferably from about 10,000 to about 80,000, and more preferably the polymer has a molecular weight of about 12,000 to about 60,000, with solvents having a miscibility in water of less than 7% by weight, optionally combined with the other solvents, providing implants intended for systemic delivery of beneficial agent having a burst index of ten or less, preferably seven or less, more preferably five or less and most preferably three or less, are particularly advantageous. The use of solvent mixtures as discussed herein can be particularly advantageous as a means of providing sufficient plasticizing of the polymer to obtain viscous gel formation and at the same time meet the desired burst indices and percentage release objectives of the compositions of the invention.

Compositions intended for local delivery of beneficial agent are formed in the same manner as those intended for systemic use. However, because local delivery of beneficial agent to a subject will not result in detectable plasma levels of beneficial agent, such systems have to be characterized by percentage of beneficial agent released in a predetermined initial period, rather than a burst index as defined herein. Most typically, that period will be the first 24 hours after implantation and the percentage will be equal to the amount by weight of the beneficial agent released in the period (e.g., 24 hours) divided by the amount by weight of the beneficial agent intended to be delivered in the duration of the delivery period, multiplied by the number 100. Compositions of the present invention will have initial bursts of 40% or less, preferably 30% or less, most preferably 20% or less, for most applications.

In many instances, it may be desirable to reduce the initial burst of beneficial agent during local administration to prevent adverse effects. For example, implants of the invention containing chemotherapeutic agents are suitable for direct injection into tumors. However, many chemotherapeutic agents may exhibit toxic side effects when administered systemically. Consequently, local administration into the tumor may be the treatment method of choice. It is necessary, however, to avoid administration of a large burst of the chemotherapeutic agent if it is possible that such agent would enter the vascular or lymphatic systems where it may exhibit side affects. Accordingly, in such instances the implantable systems of the present invention having limited burst as described herein are advantageous.

The gel formed by mixing the polymer and the solvent typically exhibits a viscosity of from about 100 to about 100,000 poise, preferably from about 500 to about 100,000 poise, more preferably from about 500 to about 100,000 poise measured at a 1.0 sec-1 shear rate and 25° C. using a Haake Rheometer at about one to two days after mixing is completed. Mixing the polymer with the solvent can be achieved with conventional low shear equipment such as a Ross double planetary mixer for from about ten minutes to about one hour, although shorter and longer periods may be chosen by one skilled in the art depending on the particular physical characteristics of the composition being prepared. Since the depot composition of the invention are administered as an injectable composition, a countervailing consideration when forming depot compositions that are viscous gels is that the polymer/solvent/beneficial agent composition have sufficiently low viscosity in order to permit it to be forced through a small diameter, e.g., 18 to 20 gauge needle. If necessary, adjustment of viscosity of the gel for injection can be accomplished with emulsifying agents as described herein. Yet, such compositions should have adequate dimensional stability so as to remain localized and be able to be removed if necessary. The particular gel or gel-like compositions of the present invention satisfy such requirements.

If the polymer composition is to be administered as an injectable gel, the level of polymer dissolution will need to be balanced with the resulting gel viscosity, to permit a reasonable force to dispense the viscous gel from a needle or a catheter, and the potential burst effect. Highly viscous gels enable the beneficial agent to be delivered without exhibiting a significant burst effect, but may make it difficult to dispense the gel through a needle or a catheter. In those instances, an emulsifying agent may optionally be added to the composition. Also, since the viscosity may generally be lowered as the temperature of the composition increases, it may be advantageous in certain applications to reduce the viscosity of the gel by heating to provide a more readily injectable composition. The shear thinning characteristics of the depot compositions of the present invention allow them to be readily injected into an animal, including humans, using standard gauge needles or catheters without requiring undue dispensing pressure.

When the emulsifying agent is mixed with the viscous gel formed from the polymer and the solvent using conventional static or mechanical mixing devices, such as an orifice mixer, the emulsifying agent forms a separate phase composed of dispersed droplets of microscopic size that typically have an average diameter of less than about 100 microns. The continuous phase is formed of the polymer and the solvent. The particles of the beneficial agent may be dissolved or dispersed in either the continuous phase or the droplet phase. In the resulting thixotropic composition, the droplets of emulsifying agent elongate in the direction of shear and substantially decrease the viscosity of the viscous gel formed from the polymer and the solvent. For instance, with a viscous gel having a viscosity of from about 5,000 to about 50,000 poise measured at 1.0 sec⁻¹ at 25° C., one can obtain a reduction in viscosity to less than 100 poise when emulsified with a 10% ethanol/water solution at 25° C. as determined by Haake Rheometer.

When used, the emulsifying agent typically is present in an amount ranging from about 5 to about 80%, preferably from about 20 to about 60% and often 30 to 50% by weight based on the amount of the implantable elastomeric depot composition, including the combined amounts of polymer, solvent, emulsifying agent and beneficial agent. Emulsifying agents include, for example, solvents that are not fully miscible with the polymer solvent or solvent mixture. Illustrative emulsifying agents are water, alcohols, polyols, esters, carboxylic acids, ketones, aldehydes and mixtures thereof. Preferred emulsifying agents are alcohols, propylene glycol, ethylene glycol, glycerol, water, and solutions and mixtures thereof. Especially preferred are water, ethanol, and isopropyl alcohol and solutions and mixtures thereof. The type of emulsifying agent affects the size of the dispersed droplets. For instance, ethanol will provide droplets that have average diameters that can be on the order of ten times larger than the droplets obtained with an isotonic saline solution containing 0.9% by weight of sodium chloride at 21° C.

It is to be understood that the emulsifying agent does not constitute a mere diluent that reduces viscosity by simply decreasing the concentration of the components of the composition. The use of conventional diluents can reduce viscosity, but can also cause the burst effect mentioned previously when the diluted composition is injected. In contrast, the implantable elastomeric depot composition of the present invention can be formulated to avoid the burst effect by selecting the appropriate polymer, the solvent and emulsifying agent so that once injected into place, the emulsifying agent has little impact on the release properties of the original system.

Although the implantable elastomeric depot compositions of the present invention preferably are formed as viscous gels, the means of administration of the implants is not limited to injection, although that mode of delivery may often be preferred. Where the implantable elastomeric depot composition will be administered as a leave-behind product, it may be formed to fit into a body cavity existing after completion of surgery or it may be applied as a flowable gel by brushing or palleting the gel onto residual tissue or bone. Such applications may permit loading of beneficial agent in the gel above concentrations typically present with injectable compositions.

C. Beneficial Agents:

The beneficial agent can be any physiologically or pharmacologically active substance or substances optionally in combination with pharmaceutically acceptable carriers and additional ingredients such as antioxidants, stabilizing agents, permeation enhancers, etc. that do not substantially adversely affect the advantageous results that can be attained by the present invention. The beneficial agent may be any of the agents which are known to be delivered to the body of a human or an animal and that are preferentially soluble in water rather than in the polymer-dissolving solvent. These agents include drug agents, medicaments, vitamins, nutrients, or the like. Included among the types of agents which meet this description are lower molecular weight compounds, proteins, peptides, genetic material, nutrients, vitamins, food supplements, sex sterilants, fertility inhibitors and fertility promoters.

Drug agents which may be delivered by the present invention include drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents including anti-inflammatory corticosteroids, antiproliferative agents, antimitotic agents, angiogenic agents, antipsychotic agents, central nervous system (CNS) agents, anticoagulants, fibrinolytic agents, growth factors, antibodies, ocular drugs, and metabolites, analogs (including synthetic and substituted analogs), derivatives (including aggregative conjugates/fusion with other macromolecules and covalent conjugates with unrelated chemical moieties by means known in the art) fragments, and purified, isolated, recombinant and chemically synthesized versions of these species.

Examples of drugs that may be delivered by the composition of the present invention include, but are not limited to, procaine, procaine hydrochloride, tetracaine, tetracaine hydrochloride, cocaine, cocaine hydrochloride, chloroprocaine, chloroprocaine hydrochloride, proparacaine, proparacaine hydrochloride, piperocaine, piperocaine hydrochloride, hexylcaine, hexylcaine hydrochloride, naepaine, naepaine hydrochloride, benzoxinate, benzoxinate hydrochloride, cyclomethylcaine, cyclomethylcaine hydrochloride, cyclomethylcaine sulfate, lidocaine, lidocaine hydrochloride, bupivacaine, bupivacaine hydrochloride, mepivacaine, mepivacaine hydrochloride, prilocaine, prilocaine hydrochloride, dibucaine and dibucaine hydrochloride, etidocaine, benzocaine, propoxycaine, dyclonin, pramoxine, oxybuprocaine, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-nor-progesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth factors such as epidermal growth factors (EGF), platelet-derived growth factors (PDGF), fibroblast growth factors (FGF), transforming growth factors-α (TGF-α), transforming growth factors-β (TGF-β), erythropoietin (EPO), insulin-like growth factor-I (IGF-I), insulin-like growth factor-II (IGF-II), interleukin-1, interleukin-2, interleukin-6, interleukin-8, tumor necrosis factor-α (TNF-α), tumor necrosis factor-β (TNF-β), Interferon-α (INF-α), Interferon-β (INF-β), Interferon-γ (INF-γ), Interferon-ω (INF-ω), colony stimulating factors (CGF), vascular cell growth factor (VEGF), thrombopoietin (TPO), stromal cell-derived factors (SDF), placenta growth factor (PIGF), hepatocyte growth factor (HGF), granulocyte macrophage colony stimulating factor (GM-CSF), glial-derived neurotropin factor (GDNF), granulocyte colony stimulating factor (G-CSF), ciliary neurotropic factor (CNTF), bone morphogenic proteins (BMP), coagulation factors, human pancreas hormone releasing factor, analogs and derivatives of these compounds, and pharmaceutically acceptable salts of these compounds, or their analogs or derivatives.

Additional examples of drugs that may be delivered by the composition of the present invention include, but are not limited to, antiproliferative/antimitotic agents including natural products such as vinca alkaloids (i.e., vinblastine, vincristine, and vinorelbine), paclitaxel, epidipodophyllotoxins (i.e., etoposide, teniposide), antibiotics (dactinomycin, actinomycin D, daunorubicin, doxorubicin and idarubicin), anthracyclines, mitoxantrone, bleomycins, plicamycin(mithramycin) and mitomycin, enzymes (L-asparaginase which systemically metabolizes L-asparagine and deprives cells which do not have the capacity to synthesize their own asparagine); antiplatelet agents such as G(GP)II_{b}IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, hirtosoureas (carmustine (BCNU) and analogs, streptozocin), trazenes—dacarbazinine (DTIC); antiproliferative/antimitotic antimetabolites, such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine (cladribine)); platinum coordination complexes (cisplatin, carboplatin), procarbazine, hydroxyurea, mitotane, aminoglutethimide; hormones (i.e., estrogen); antipsychotic agents (such as antipsychotic drugs, neuroleptic drugs, tranquillizers and antipsychotic agents binding to dopamine, histamine, muscarinic cholinergic, adrenergic and serotonin receptors, including, but not limited to, phenothiazines, thioxanthenes, butyrophenones, dibenzoxazepines, dibenzodiazepines, diphenylbutylpiperidines, risperdone, paliperidone and the like); CNS agents; anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory, such as adrenocortical steroids (cortisol, cortisone, fluorocortisone, prednisone, prednisolone, 6α-methylprednisolone, triamcinolone, betamethasone, and dexamethasone), non-steroidal agents (salicylic acid derivatives, i.e., aspirin; para-aminophenol derivatives, i.e., acetaminophen); indole and indene acetic acids (indomethacin, sulindac, and etodolac), heteroaryl acetic acids (tolmetin, diclofenac, and ketorolac), arylpropionic acids (ibuprofen and derivatives), anthranilic acids (mefenamic acid and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives (cyclosporine, tacrolimus (FK-506), sirolimus (rapamycin), azathioprine, mycophenolate mofetil); angiogenic agents, vascular endothelial growth factor (VEGF), fibroblast growth factor (FGF); angiotensin receptor blocker; nitric oxide donors; anti-sense oligonucleotides and combinations thereof; cell cycle inhibitors, mTOR inhibitors, and growth factor signal transduction kinase inhibitors, analogs and derivatives of these compounds, and pharmaceutically acceptable salts of these compounds, or their analogs or derivatives.

In certain preferred embodiments, the beneficial agent includes chemotactic growth factors, proliferative growth factors, stimulatory growth factors, and transformational peptide growth factors including genes, precursors, post-translational-variants, metabolites, binding-proteins, receptors, receptor agonists and antagonists of the following growth factor families: epidermal growth factors (EGFs), platelet-derived growth factor (PDGFs), insulin-like growth factors (IGFs), fibroblast-growth factors (FGFs), transforming-growth factors (TGFs), interleukins (ILs), colony-stimulating factors (CSFs, MCFs, GCSFs, GMCSFs), Interferons (IFNs), endothelial growth factors (VEGF, EGFs), erythropoietins (EPOs), angiopoietins (ANGs), placenta-derived growth factors (PIGFs), and hypoxia induced transcriptional regulators (HIFs).

The present invention also finds application with chemotherapeutic agents for the local application of such agents to avoid or minimize systemic side effects. Gels of the present invention containing chemotherapeutic agents may be injected directly into the tumor tissue for sustained delivery of the chemotherapeutic agent over time. In some cases, particularly after resection of the tumor, the gel may be implanted directly into the resulting cavity or may be applied to the remaining tissue as a coating. In cases in which the gel is implanted after surgery, it is possible to utilize gels having higher viscosities since they do not have to pass through a small diameter needle. Representative chemotherapeutic agents that may be delivered in accordance with the practice of the present invention include, for example, carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986. The present application has particular utility in the sustained delivery of water soluble chemotherapeutic agents, such as, for example, cisplatin and carboplatin and the water soluble derivatives of paclitaxel. Those characteristics of the invention that minimize the burst effect are particularly advantageous in the administration of water soluble beneficial agents of all kinds, but particularly those compounds that are clinically useful and effective but may have adverse side effects.

To the extent not mentioned above, the beneficial agents described in aforementioned U.S. Pat. No. 5,242,910 can also be used. One particular advantage of the present invention is that materials, such as proteins, as exemplified by the enzyme lysozyme, and cDNA, and DNA incorporated into vectors both viral and nonviral, which are difficult to microencapsulate or process into microspheres can be incorporated into the compositions of the present invention without the level of degradation caused by exposure to high temperatures and denaturing solvents often present in other processing techniques.

The beneficial agent is preferably incorporated into the viscous gel formed from the polymer and the solvent in the form of particles typically having an average particle size of from about 0.1 to about 250 microns, preferably from about 1 to about 125 microns and often from 10 to 90 microns. For instance, particles having an average particle size of about 5 microns have been produced by spray drying or freeze drying an aqueous mixture containing 50% sucrose and 50% chicken lysozyme (on a dry weight basis) and mixtures of 10-20% hGH and 15-30 mM zinc acetate. Such particles have been used in certain of the examples illustrated in the figures. Conventional lyophilization processes can also be utilized to form particles of beneficial agents of varying sizes using appropriate freezing and drying cycles, followed by appropriate grounding and sieving.

To form a suspension or dispersion of particles of the beneficial agent in the viscous gel formed from the polymer and the solvent, any conventional low shear device can be used, such as a Ross double planetary mixer at ambient conditions. In this manner, efficient distribution of the beneficial agent can be achieved substantially without degrading the beneficial agent.

The beneficial agent is typically dissolved or dispersed in the composition in an amount of from about 0.1 to about 70% by weight, preferably in an amount of from about 0.5 to about 50% and often 1 to 30% by weight of the combined amounts of the polymer, solvent and beneficial agent. Depending on the amount of beneficial agent present in the composition, one can obtain different release profiles and burst indices. More specifically, for a given polymer and solvent, by adjusting the amount of these components and the amount of the beneficial agent, one can obtain a release profile that depends more on the degradation of the polymer than the diffusion of the beneficial agent from the composition or vice versa. In general, during the early stages, the release rate profile is generally controlled by the rate of diffusion and the rate of dissolution of the beneficial agent from the composition; while in the later stages, polymer degradation is the major factor in determining the release rate profiles. In this respect, at lower beneficial agent loading levels, the release rate profile depends primarily on the rate of degradation of the polymer, and secondarily on the diffusion of the beneficial agent from the composition, wherein generally the release rate increases or is constant (e.g., flat profile) with time.

At higher beneficial agent loading levels, the release rate depends on the solubility of the beneficial agent in the depot composition or surrounding medium. For example, if the beneficial agent has the high solubility in the composition or surrounding medium, the release profile depends primarily on the rate of diffusion of the beneficial agent from the composition and secondarily on the rate of polymer degradation, wherein generally, the release rate decreases with time. If the beneficial agent has very low solubility in the composition or surrounding medium, the release profile depends primarily on the rate of diffusion and the rate of dissolution of the beneficial agent from the composition, and secondarily on the rate of polymer degradation, wherein generally the release rate is constant with time.

At intermediate beneficial agent loading levels, the release rate depends on the combined effects of diffusion of the beneficial agent from the composition and the rate of polymer degradation, wherein this combined effect can be tailored to achieve a substantially constant release rate profile. In order to minimize burst, loading of beneficial agent on the order of 30% or less by weight of the overall gel composition, i.e., polymer, solvent and beneficial agent, is preferred, and loading of 20% or less is more preferred.

Release rates and loading of beneficial agent will be adjusted to provide for therapeutically-effective delivery of the beneficial agent over the intended sustained delivery period. Preferably, the beneficial agent will be present in the polymer gel at concentrations that are above the saturation concentration of beneficial agent in water to provide a drug reservoir from which the beneficial agent is dispensed. While the release rate of beneficial agent depends on the particular circumstances, such as the beneficial agent to be administered, release rates on the order of from about 0.1 to about 10,000 micrograms/day, preferably from about 1 to about 5,000 micrograms per day, for periods of from about one week to about one year can be obtained. Greater amounts may be delivered if delivery is to occur over shorter periods. Generally, higher release rate is possible if a greater burst can be tolerated. In instances where the gel composition is surgically implanted, or used as a “leave behind” depot when surgery to treat the disease state or another condition is concurrently conducted, it is possible to provide higher doses that would normally be administered if the implant was injected. Further, the dose of beneficial agent may be controlled by adjusting the volume of the gel implanted or the injectable gel injected.

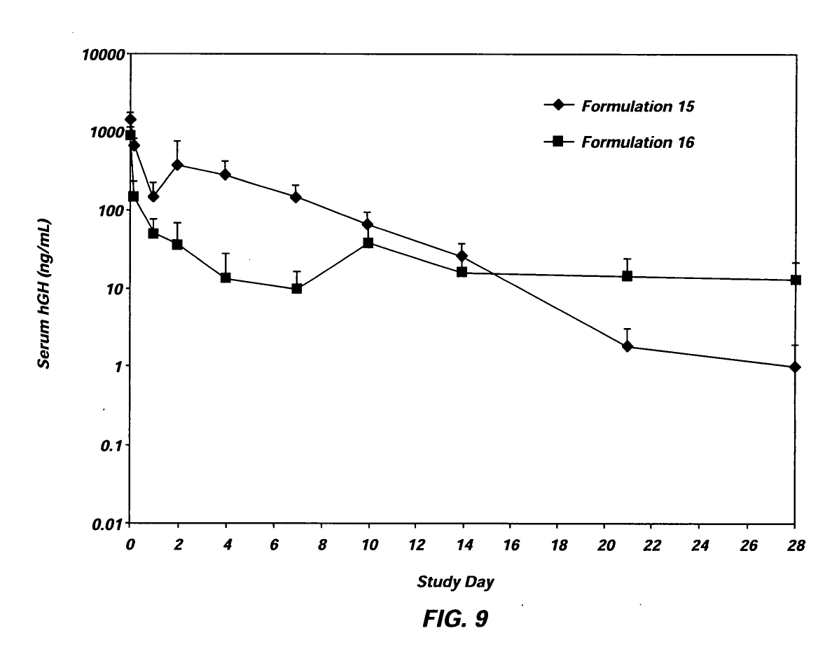

FIG. 9 illustrates representative release profiles of hGH obtained in rats from preferred compositions of this invention. As illustrated in the figures, the implantable elastomeric depot gel formulations of the invention comprising polymers provide a controlled, sustained release of a beneficial agent over a specified/desired duration of time. The duration and the release rate profiles can be adjusted depending on the nature of the polymer and the properties of the polymer (e.g., MW, comonomer ratios, end-group), the nature of the solvent and the polymer/solvent ratio.

D. Optional Additional Components:

Other components may be present in the implantable elastomeric depot composition, to the extent they are desired or provide useful properties to the composition, such as polyethylene glycol, hydroscopic agents, stabilizing agents, pore forming agents, thixotropic agents and others. When the composition includes a peptide or a protein that is soluble in or unstable in an aqueous environment, it may be highly desirable to include a solubility modulator that may, for example, be a stabilizing agent, in the composition. Various modulating agents are described in U.S. Pat. Nos. 5,654,010 and 5,656,297, which are incorporated herein by reference. In the case of hGH, for example, it is preferable to include an amount of a salt of a divalent metal, preferably zinc. Examples of such modulators and stabilizing agents, which may form complexes with the beneficial agent or associate to provide the stabilizing or modulated release effect, include metal cations, preferably divalent, present in the composition as magnesium carbonate, zinc carbonate, calcium carbonate, magnesium acetate, magnesium sulfate, zinc acetate, zinc sulfate, zinc chloride, magnesium chloride, magnesium oxide, magnesium hydroxide, other antacids, and the like. The amounts of such agents used will depend on the nature of the complex formed, if any, or the nature of the association between the beneficial agent and the agent. Molar ratios of solubility modulator or stabilizing agent to beneficial agent of about 100:1 to 1:1, preferably 10:1 to 1:1, typically can be utilized.

The thixotropic agent, i.e., an agent that imparts thixotropic properties to the polymer gel, is selected from the lower alkanols. Lower alkanol means an alcohol that contains 2-6 carbon atoms and is straight chain or branched chain. Such alcohols may be exemplified by ethanol, isopropanol, and the like. Importantly, such a thixotropic agent is not a polymer solvent. (See, e.g., Development of an in situ forming biodegradable poly-lactide-co-glycolide system for controlled release of proteins, Lambert, W. J., and Peck, K. D., Journal of Controlled Release, 33 (1995) 189-195.)

Pore forming agents include biocompatible materials that, when contacted with body fluids, dissolve, disperse or degrade to create pores or channels in the polymer matrix. Typically, organic and non-organic materials that are water soluble, such as sugars (e.g., sucrose and dextrose), water soluble salts (e.g., sodium chloride, sodium phosphate, potassium chloride, and sodium carbonate), water soluble solvents, such as N-methyl-2-pyrrolidone and polyethylene glycol, and water soluble polymers (e.g., carboxmethylcellulose, hydroxypropylcellulose, and the like) can conveniently be used as pore formers. Such materials may be present in amounts varying from about 0.1% to about 100% of the weight of the polymer, but will typically be less than 50% and more typically be less than 10-20% of the weight of the polymer.

II. Utility and Administration:

The means of administration of the depot compositions is not limited to injection, although that mode of delivery may often be preferred. Where the depot composition will be administered as a leave-behind product, it may be formed to fit into a body cavity existing after completion of surgery or it may be applied as a flowable gel by brushing or palleting the gel onto residual tissue or bone. Such applications may permit loading of beneficial agent in the gel above concentrations typically present with injectable compositions.

Compositions of this invention without beneficial agent are useful for wound healing, bone repair and other structural support purposes.

To further understand the various aspects of the present invention, the results set forth in the previously described figures were obtained in accordance with the following examples.

### EXAMPLE 1

### Synthesis of Poly(ε-caprolactone-co-glycolide-co-l,lactide)(PCL-GA-I, LA) 40:55:5

Synthesis of Low Molecular Weight PCL-GA-I, LA

In the glove box, 168 μL (55 μmol) of a 0.33 M stannous octoate solution in toluene (Ethicon Inc., Cornelia, Ga., USA), 5.31 grams (50 mmol) of diethylene glycol (Fluka Chemical Co., Milwaukee, Wis., USA), 156.7 grams (1.35 mol) of glycolide (Noramco, Inc., Athens, Ga., USA), 117.0 grams (1.025 mol) of caprolactone (Union Carbide Corp., Danbury, Conn., USA), and 18.0 grams (0.125 mol) I-lactide (Purac America, Lincolnshire, Ill., USA) were transferred into a flame dried, 500 mL round bottom flask equipped with a stainless steel mechanical stirrer and a nitrogen gas blanket. The reaction flask was placed in a room temperature oil bath, heated to 190° C. and then held at 190° C. for 16 hours. The reaction was allowed to cool to 80° C., then poured out of the flask into a clean dry polypropylene jar. The terpolymer was then vacuum dried overnight at room temperature. No de-volatilization step was necessary. The inherent viscosity was measured and found to be 0.35 dL/g in HFIP at 25° C. (c=0.1 g/dL). Polymer composition by ¹H NMR: 42.9% PCL, 52.3% PGA, 4.4% PLA, <0.2% glycolide, <0.2% ε-caprolactone, and <0.2% I-lactide. Gel Permeation Chromatogram (GPC) determined the molecular weight of M_{w}=13600, Mₙ=9000, PDI=1.5 using poly(methyl methacrylate) standards in THF.

Synthesis of Intermediate Molecular Weight PCL-GA-I,LA

In the glove box, 335 μL (111 μmol) of a 0.33 M stannous octoate solution in toluene (Ethicon Inc., Cornelia, Ga., USA), 5.31 grams (50 mmol) of diethylene glycol (Fluka Chemical Co., Milwaukee, Wis., USA), 313.4 grams (2.70 mol) of glycolide (Noramco, Inc., Athens, Ga., USA), 234.0 grams (2.05 mol) of F-caprolactone (Union Carbide Corp., Danbury, Conn., USA), and 36.1 grams (0.25 mol) I-lactide (Purac America, Lincolnshire, Ill., USA) were transferred into a flame dried, 1000 mL round bottom flask equipped with a stainless steel mechanical stirrer and a nitrogen gas blanket. The reaction flask was placed in a room temperature oil bath, heated to 190° C., and then held at 190° C. for 16 hours. The reaction was allowed to cool to room temperature overnight. The terpolymer was isolated from the reaction flask by freezing in liquid nitrogen and breaking the glass. Any remaining glass fragments were removed from the terpolymer using a bench grinder. The terpolymer was again frozen with liquid nitrogen and broken off the mechanical stirring paddle and allowed to warm to room temperature in a vacuum oven overnight. No de-volatilization step was necessary. The inherent viscosity was measured and found to be 0.53 dL/g in HFIP at 25° C. (c=0.1 g/dL). Polymer composition by ¹H NMR: 40.2% PCL, 53.9% PGA, 5.7% PLA, 0.2% glycolide, <0.2% ε-caprolactone, and <0.2% l-lactide. Gel Permeation Chromatogram (GPC) determined the molecular weight of M_{w}=23400, Mₙ=16400, PDI=1.4 using poly(methyl methacrylate) standards in THF.

Synthesis of High Molecular Weight PCL-GA-I,LA

In the glove box, 84 μL (28 μmol) of a 0.33 M stannous octoate solution in toluene (Ethicon Inc., Cornelia, Ga., USA), 119 μL (1.25 mmol) of diethylene glycol (Fluka Chemical Co., Milwaukee, Wis., USA), 78.35 grams (675 mmol) of glycolide (Noramco, Inc., Athens, Ga., USA.), 58.5 grams (513 mmol) of ε-caprolactone (Union Carbide Corp., Danbury, Conn., USA), and 9.0 grams (0.625 mol) I-lactide (Purac America, Lincolnshire, Ill., USA) were transferred into a flame dried, 250 mL round bottom flask equipped with a stainless steel mechanical stirrer and a nitrogen gas blanket. The reaction flask was placed in a room temperature oil bath, heated to 190° C., and then held at 190° C. for 16 hours. The reaction was allowed to cool to room temperature overnight. The terpolymer was isolated from the reaction flask by freezing in liquid nitrogen and breaking the glass. Any remaining glass fragments were removed from the terpolymer using a bench grinder. The terpolymer was again frozen with liquid nitrogen and broken off the mechanical stirring paddle and allowed to warm to room temperature in a vacuum oven overnight. The terpolymer was added to an aluminum pan and then de-volatilized under vacuum at 90° C. for 54 hours. The inherent viscosity was measured and found to be 1.41 dL/g in HFIP at 25° C. (c=0.1 g/dL). Polymer composition by ¹H NMR: 38.4% PCL, 55.3% PGA, 5.3% PLA, <0.2% glycolide, 0.9% c-caprolactone, and <0.2% l-lactide. Gel Permeation Chromatogram (GPC) determined the molecular weight of M_{w}=62000, Mₙ=33500, PDI=1.8 using poly(methyl methacrylate) standards in THF.

### EXAMPLE 2

### Synthesis of Poly (ε-caprolactone-co-glycolide-co-d,l,lactide)(PCL-GA-dl, LA) 40:55:5

In the glove box, 168 μL (55 μmol) of a 0.33 M stannous octoate solution in toluene (Ethicon Inc., Cornelia, Ga., USA), 2.65 grams (25 mmol) of diethylene glycol (Fluka Chemical Co., Wis., USA), 156.7 grams (1.35 mol) of glycolide (Noramco, Inc., Athens, Ga., USA), 117.0 grams (1.025 mol) of F-caprolactone (Union Carbide Corp., Danbury, Conn., USA), and 18.0 grams (0.125 mol) d,l-lactide (Purac America, Lincolnshire, Ill., USA) were transferred into a flame dried, 500 mL round bottom flask equipped with a stainless steel mechanical stirrer and a nitrogen gas blanket. The reaction flask was placed in a room temperature oil bath, heated to 190° C. and then held at 190° C. for 16 hours. The reaction was allowed to cool to room temperature overnight. The terpolymer was isolated from the reaction flask by freezing in liquid nitrogen and breaking the glass. Any remaining glass fragments were removed from the terpolymer using a bench grinder. The terpolymer was again frozen with liquid nitrogen and broken off the mechanical stirring paddle and allowed to warm to room temperature in a vacuum oven overnight. No de-volatilization step was necessary. The inherent viscosity was measured and found to be 0.56 dL/g in HFIP at 25° C. (c=0.1 g/dL). Polymer composition by ¹H NMR: 41.8% PCL, 53.1% PGA, 4.7% dI-PLA, ≦0.2% glycolide, <0.2% ε-caprolactone, and ≦0.2% dl-lactide. Gel Permeation Chromatogram (GPC) determined the molecular weight of M_{w}=24000, Mₙ=14500, PDI=1.6 using poly(methyl methacrylate) standards in THF.

### EXAMPLE 3

### Differential Scanning Calorimeter (DSC) Measurements

The glass transition temperature (Tg) of PCL-GA-LA and PLGA RG502 used in the present invention was determined using a differential scanning calorimeter (DSC) (Perkin Elmer PYRIS Diamond DSC, Shelton, Conn.). The DSC sample pan was tared on a Mettler PJ3000 top loader balance. About 10 to 20 mg of polymer sample was placed in the pan. The weight of the sample was recorded. The DSC pan cover was positioned onto the pan and a presser was used to seal the pan. The temperature was scanned in 10° C. increments from −60° C. to 90° C.

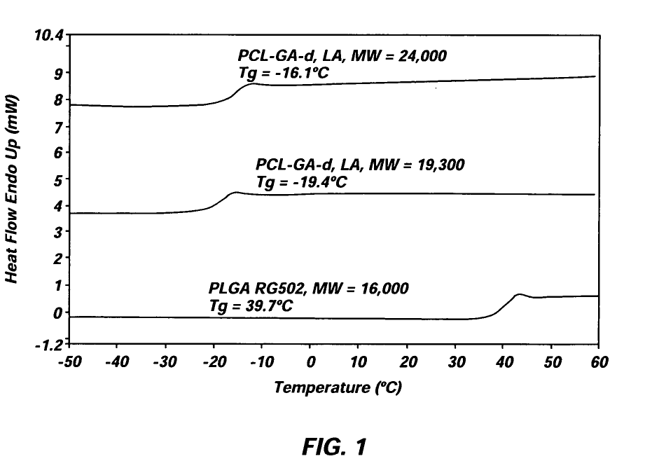

FIG. 1 compares the DSC diagrams of PCL-GA-LA copolymers with either l-lactic acid or di-lactic acid and PLGA RG502 used in the formulations presented in this invention. Those data indicate that the PCL containing copolymers used in this invention had the glass transition temperatures (“Tg”) below 0° C. as opposed to ca. 40° C. for PLGA RG502, illustrating that the PCL containing copolymers are certainly in their robber state at or near body temperature.

### EXAMPLE 4

### Depot Vehicle Preparation

A gel vehicle for use in an implantable elastomeric depot of the composition was prepared as follows. A glass vessel was tared on a Mettler PJ3000 top loader balance. Poly(D,L-lactide-co-glycolide) (PLGA), available as 50:50 Resomer® RG502 (PLGA RG502), or polycaprolactone-glycolic acid-L, lactic acid) (PCL-GA-LA) synthesized as described in the examples 1 and 2 above, was weighed and dispensed into a Keyence hybrid mixer bowl (made of HD polyethylene). The mixing bowl was tightly sealed, placed into the Keyence hybrid mixer (model HM-501, Keyence, Japan), and mixed for five to ten minutes at mixing speed (revolution 2000 rpm and rotation 800 rpm).

Additional depot gel vehicles are prepared with the following solvents or mixtures: benzyl benzoate (“BB”), benzyl alcohol (“BA”), and ethyl benzoate (“EB”), triactin, ethyl oleate, lauryl lactate and the following polymers: Poly (L-lactide) Resomer® L104, PLA-L104, Poly(D,L-lactide-co-glycolide) 50:50 Resomer® RG502, Poly(D,L-lactide-co-glycolide) 50:50 Resomer® RG502H, Poly(D,L-lactide-co-glycolide) 50:50 Resomer® RG503, Poly(D,L-lactide-co-glycolide) 50:50 Resomer® RG755, Poly L-Lactide (Resomer® L206, Resomer® L207, Resomer® L209, Resomer® L214); Poly D,L Lactide (Resomer® R104, Resomer® R202, Resomer® R203, Resomer® R206, Resomer® R207, Resomer® R208); Poly L-Lactide-co-D,L-lactide 90:10 (Resomer® LR209); Poly D-L-lactide-co-glycolide 75:25 (Resomer® RG752, Resomer® RG756); Poly D,L-lactide-co-glycolide 85:15 (Resomer® RG858); Poly L-lactide-co-trimethylene carbonate 70:30 (Resomer® LT706); Poly dioxanone (Resomer® X210) (Boehringer Ingelheim Chemicals, Inc., Petersburg, Va.); DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L. P., Cincinatti, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.). Additional examples of polymers useful in this invention are described in U.S. Pat. Nos. 6,113,624; 5,868,788; 5,714,551; 5,713,920; 5,639,851 and 5,468,253. Typical polymer molecular weights were in the range of 14,000-80,000 (M_{w}). Representative gel vehicles are described in Tables 1-3 below.

### EXAMPLE 5

### Viscosity and Injection Force Measurement of Depot Gel Formulations

Viscosity of the depot vehicle formulations was tested using a Bohlin CVO 120 Rhoemeter. All tests were performed at 24° C. using 20 mm parallel plates. The injection force of the depot vehicle formulations was tested on an Instron tensile testing instrument, where the maximum force required to move the syringe plunger at a speed of 1 ml/minute was determined. The vehicle formulations were pre-filled into Hamilton syringes prior to the Instron tests. All tests were conducted at room temperature, using a 24-gauge 0.5 inch long needle.

### EXAMPLE 6

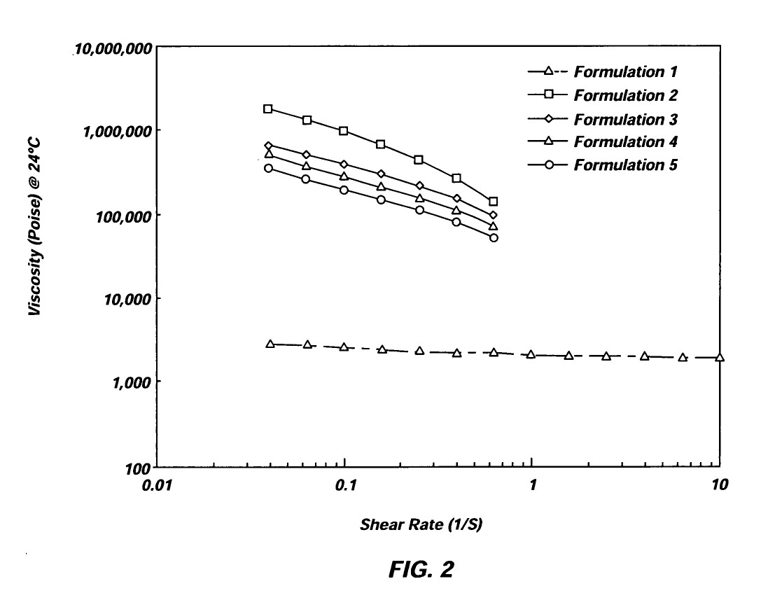

Rheological behavior for depot vehicles formulated with the solvent benzyl benzoate (BB), benzyl alcohol (BA) or mixtures thereof as described in this invention was performed. The vehicle formulations comprising 50 wt. % of PCL-GA-LA (CL/G/L) copolymer in the different solvents (BB, BA or mixtures thereof) (e.g., formulations 2-5), respectively, were prepared according to the procedures outlined in Example 4. For comparative purposes, vehicle formulations comprising only PLGA RG502 in BB (e.g., formulation 1) was also prepared. Table 1 lists the formulations used in the test. Formulations 1-5 were tested for viscosity under various shear rates. As indicated in FIG. 2, significantly higher viscosity and shear thinning behaviour was observed when PCL-GA-LA was used as the polymer in different solvents (e.g., formulations 2-5), as compared to the formulation using PLGA RG502 in BB (e.g., formulation 1).

TABLE 1

| | Polymer | Benzyl Benzoate | Benzyl Alcohol |
|---|---|---|---|
| Formulation | (wt. %) | (wt. %) | (wt. %) |
| 1 | 50.0^{a} | 50.0 | 0.0 |
| 2 | 50.0^{b} | 50.0 | 0.0 |
| 3 | 50.0^{b} | 37.5 | 12.5 |
| 4 | 50.0^{b} | 25.0 | 25.0 |
| 5 | 50.0^{b} | 0 | 50 |

^{a}= PLGA RG502, MW = 16,000;
^{b}= PCL-GA-LA (40-55-5), MW = 30,600.

### EXAMPLE 7

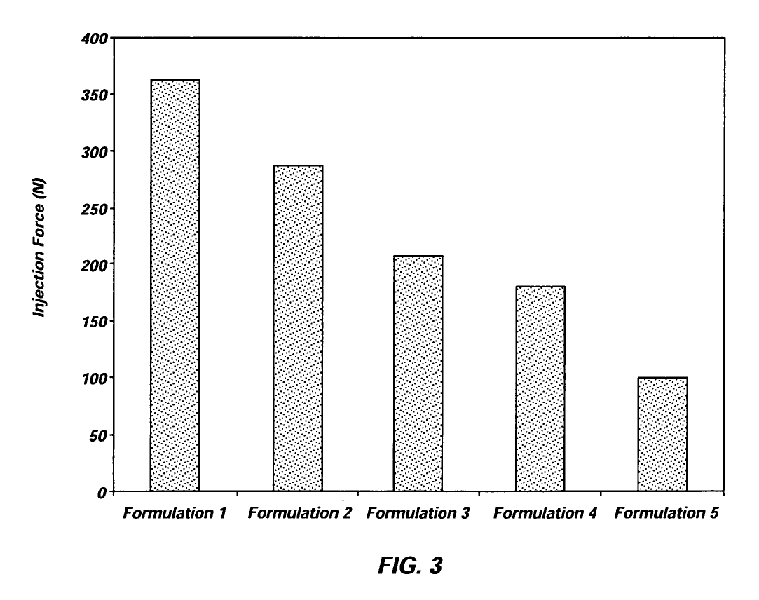

The injection force required to dispense depot vehicles was evaluated for the formulations tabulated in Table 1. The formulations were injected through a 24-gauge needle at 1 ml/minute, at room temperature. As indicated in FIG. 3, significantly reduced injection force was observed when PCL-GA-LA was used as the polymer in different solvents (e.g., formulations 2-5), in contrast to formulations using PLGA RG502 in BB (e.g., formulation 1). Notably, due to shear thinning behavior, even though much higher molecular weight of PCL-GA-LA copolymer was used, the formulations using PCL-GA-LA copolymer in various solvents (e.g., formulations 2-5), showed significantly reduced injection force while maintaining viscosities equal to or greater than the formulations using PLGA RG502 polymer (e.g., formulation 1), at lower shear rate, thus maintaining the intactness of the depot after injection into the animals.

### EXAMPLE 8

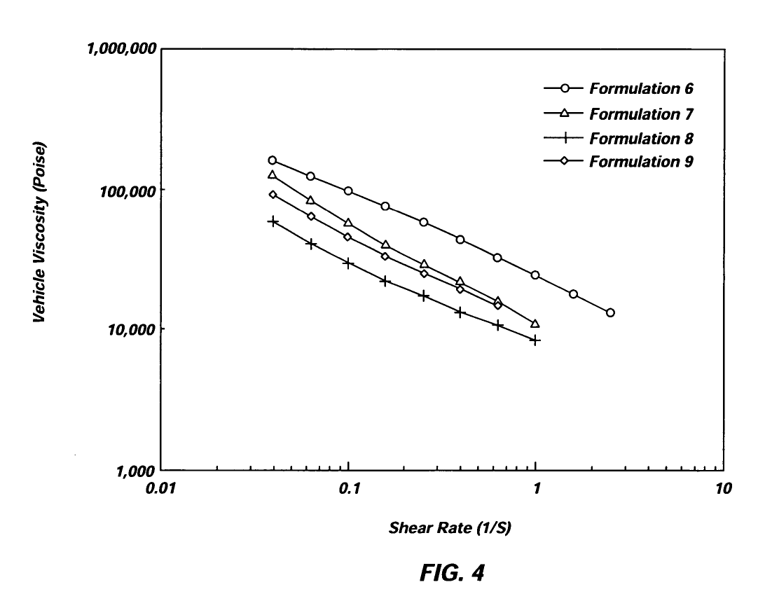

Rheological behavior for depot vehicles formulated with various PCL-GA-LA polymers having various molecular weights and BB as prepared according to this invention was performed. The vehicle formulations comprising 30 wt. % of PCL-GA-LA having varying molecular weights and 70 wt. % of BB were prepared according to the procedures outlined in Example 4 and are tabulated in Table 2 below. Formulations 6-9 were tested for viscosity under various shear rates. As illustrated in FIG. 4, all formulations showed significant shear thinning behavior independent of the molecular weight of the polymer.

TABLE 2

| | | | Benzyl Benzoate |
|---|---|---|---|
| Formulation | Polymer(MW)^{a} | Polymer (wt. %) | (wt. %) |
| 6 | 60,400 | 30.0 | 70.0 |
| 7 | 30,600 | 30.0 | 70.0 |
| 8 | 19,400 | 30.0 | 70.0 |
| 9 | 22,600 | 30.0 | 70.0 |

^{a}= PCL-GA-LA (40-55-5)

### EXAMPLE 9

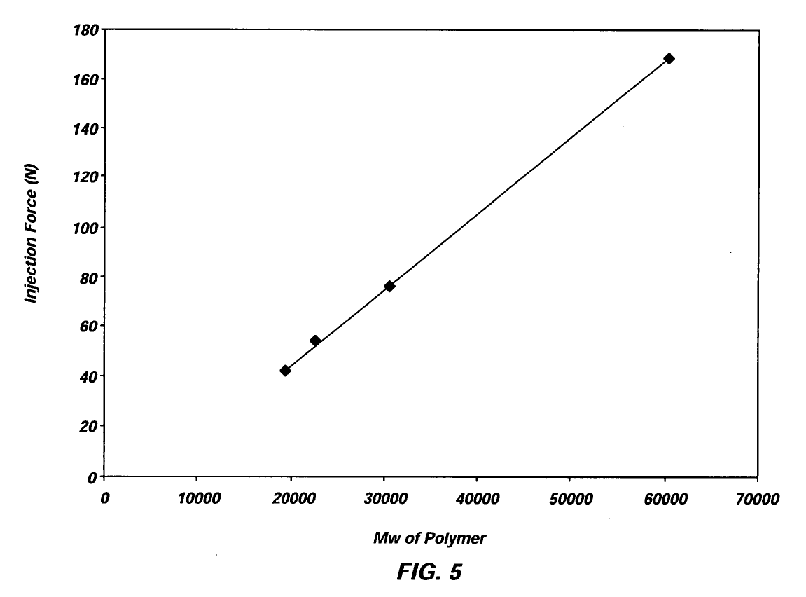

The injection force required to dispense depot vehicles was evaluated for the formulations tabulated in Table 2. The formulations were injected through a 24-gauge needle at 1 ml/minute, at room temperature. As illustrated in FIG. 5, there was a linear correlation between the injection force and molecular weight of the polymer, indicating that the injection force of the formulations can be easily adjusted by tailoring the molecular weight of the polymer.

### EXAMPLE 10

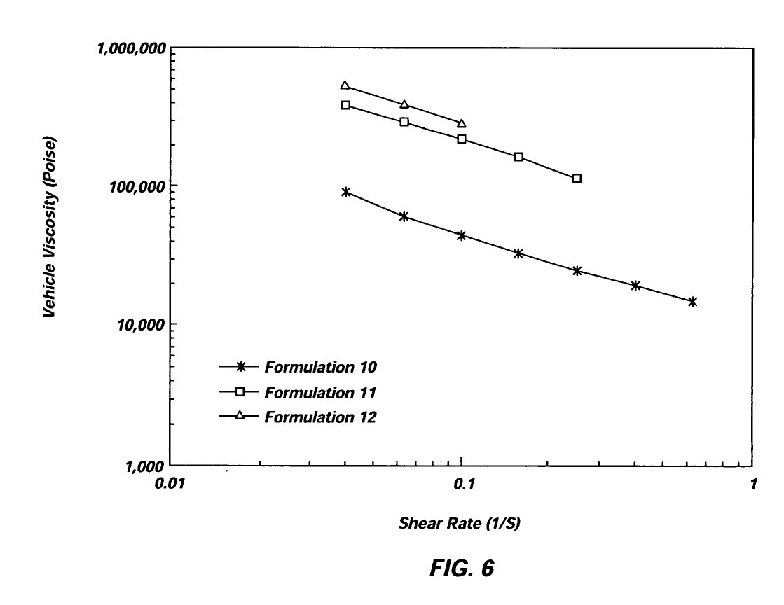

Depot vehicles formulations of the invention having various polymer/solvent ratios, wherein the polymer is PCL-GA-LA (MW=22,400) and the solvent is benzyl benzoate, were prepared according to the procedures outlined in Example 4 and are tabulated in Table 3. These formulations 10-12) were tested for viscosity under various shear rates. As illustrated in FIG. 6, regardless of the various polymer/solvent ratios, all formulations showed significant shear thinning behavior.

TABLE 3

| | Polymer^{a} | Benzyl Benzoate |
|---|---|---|
| Formulation | (wt. %) | (wt. %) |
| 10 | 30.0 | 70.0 |
| 11 | 40.0 | 60.0 |
| 12 | 45.0 | 55.0 |

^{a}= PCL-GA-LA (40-55-5), MW = 22,400.

### EXAMPLE 11

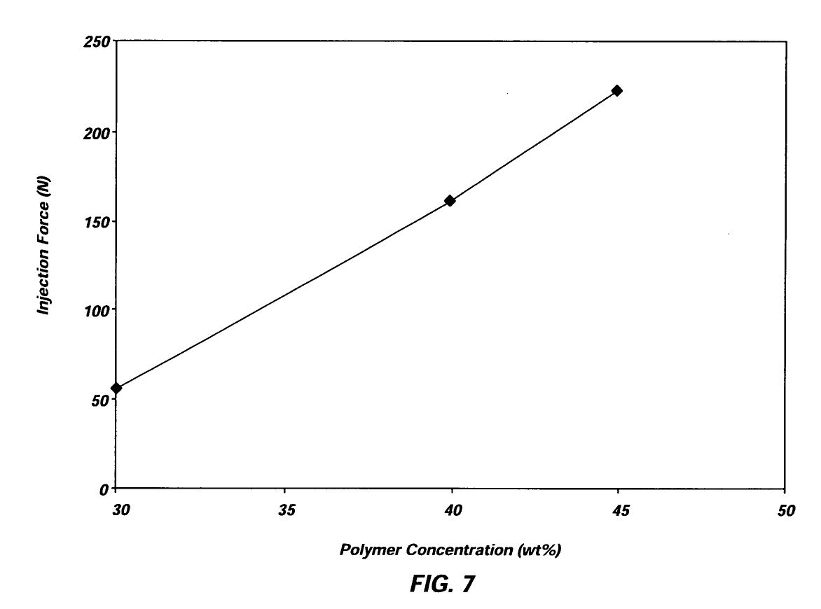

The injection force required to dispense depot vehicles was evaluated for the formulations indentified Example 10. The formulations were injected through a 24-gauge needle at 1 ml/minute, at room temperature. As illustrated in FIG. 7, the injection force of formulations increased with the increase in the proportion of the polymer within the vehicle composition. Thus, the injection force of the formulations can be adjusted by tailoring the polymer/solvent ratios.

### EXAMPLE 12

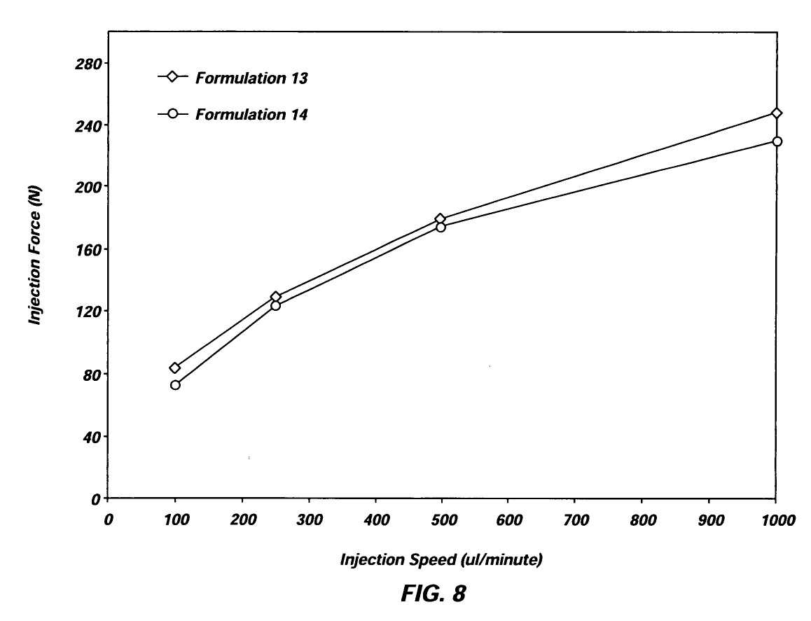

The vehicle formulations comprising PCL-GA-LA copolymers having either l-lactic acid or dl-lactic acid in the terpolymers with similar molecular weight of approximately 22,400 to approximately 23,500 in benzyl benzoate (BB) were prepared according to the procedures outlined in Example 4. The injection force required to dispense depot vehicles formulations identified in Table 4 was evaluated. The formulations were injected through a 24-gauge needle at various speeds, at room temperature. As illustrated in FIG. 8, terpolymers with l-lactic acid and dl-lactic acid had similar injection forces. It is worth noting that the increase in injection force of the formulations at higher injection speeds is much lower in magnitude as compared to the increase in injection force at lower injection speeds, indicating the shear thinning reduces the injection force.

TABLE 4

| | | | | Benzyl |
|---|---|---|---|---|
| | | | Polymer | Benzoate |
| Formulation | Polymer | MW | (wt. %) | (wt. %) |
| 13 | PCL-GA-I,LA | 22,400 | 45.0 | 55.0 |
| 14 | PCL-GA-dlLA | 23,500 | 45.0 | 55.0 |

### EXAMPLE 13

### hGH Particle Preparation

Human growth hormone (hGH) particles (optionally containing zinc acetate) were prepared as follows:

hGH solution (5 mg/ml) solution in water (BresaGen Corporation, Adelaide, Australia) was concentrated to 10 mg/mL using a Concentration/Dialysis Selector diafiltering apparatus. The diafiltered hGH solution was washed with five times volume of tris or phosphate buffer solution (pH 7.6). Particles of hGH were then formed by spray drying or lyophilization using conventional techniques. Phosphate buffer solutions (5 or 50 mM) containing hGH (5 mg/mL) (and optionally various levels of zinc acetate (0 to 30 mM) when Zn complexed particles were prepared) were spray-dried using a Yamato Mini Spray dryer set at the following parameters:

| Spray Dryer Parameter | Setting |
|---|---|
| Atomizing Air | 2 psi |
| Inlet Temperature | 120° C. |
| Aspirator Dial | 7.5 |
| Solution Pump | 2-4 |
| Main Air Valve | 40-45 psi |

Lyophilized particles were prepared from tris buffer solutions (5 or 50 mM: pH 7.6) containing hGH (5 mg/mL) using a Durastop μP Lyophilizer in accordance with the following freezing and drying cycles:

| | |
|---|---|
| Freezing cycle | Ramp down at 2.5° C./minute to −30° C. and hold for 30 |
| | minutes |
| | Ramp down at 2.5° C./minute to −30° C. and hold for 30 |
| | minutes |
| Drying cycle | Ramp up at 0.5° C./minute to 10° C. and hold for 960 |
| | minutes |
| | Ramp up at 0.5° C./minute to 20° C. and hold for 480 |
| | minutes |
| | Ramp up at 0.5° C./minute to 25° C. and hold for 300 |
| | minutes |
| | Ramp up at 0.5° C./minute to 30° C. and hold for 300 |
| | minutes |
| | Ramp up at 0.5° C./minute to 5° C. and hold for 5000 |
| | minutes |

Lyophilized hGH formulation was grounded and sieved through a 70 mesh screen followed by a 400 mesh screen to obtain particles having a size range between 38-212 microns.

### EXAMPLE 14

### Drug Loading

Sieved particles comprising beneficial agent prepared as above are added to a gel vehicle in an amount of 10-20% by weight and blended manually until the dry powder is wetted completely. Then, the milky light yellow particle/gel mixture is thoroughly blended by conventional mixing using a Caframo mechanical stirrer with an attached square-tip metal spatula. Resulting formulations are illustrated in Table 5 below. Final homogenous gel formulations were transferred to 3, 10 or 30 cc disposable syringes for storage or dispensing.

A representative number of implantable gels were prepared in accordance with the foregoing procedures and tested for in vitro release of beneficial agent as a function of time and also in vivo studies in rats to determine release of the beneficial agent as determined by blood serum or plasma concentrations of beneficial agent as a function of time.

TABLE 5

| | | | Polymer | BB | Ethanol |
|---|---|---|---|---|---|
| Formulation^{a} | Polymer Type | MW | (wt. %) | (wt. %) | (wt. %) |
| 15 | PLGA RG502 | 16,000 | 39.6 | 49.5 | 0.9 |
| 16 | PCL-GA-1, LA | 19,400 | 40.5 | 49.5 | NA |

^{a}10 wt. % hGH particle loading.

### EXAMPLE 15

### hGH In Vivo Studies

In vivo studies in rats were performed following an open protocol to determine serum levels hGH upon systemic administration of hGH via the implant systems of this invention. Depot gel hGH formulations were loaded into customized 0.5 cc disposable syringes. Disposable 18 gauge 1″ needles were attached to the syringes and were heated to 37° C. using a circulator bath. Depot gel hGH formulations were injected into immunosuppressed rats and serum samples were collected post-injection at one hour, four hours, days 1, 2, 4, 7, 10, 14, 21 and 28. All serum samples were stored at 4° C. prior to analysis. Samples were analyzed for intact hGH content using a radio immunoassay (RIA). At the end of study the rats were euthanized for gross clinical observation and the depot was retrieved for intactness observations.

FIG. 9 illustrates representative in vivo release profiles of human growth hormone (“hGH”) obtained in rats from various depot compositions, including those of the present invention. The in vivo release profile of the depot formulation with PCL-GA-LA copolymer is comparable to or even better than the control formulation (using PLGA RG502).

Thus, the depot compositions of the present invention have desirable elastomeric properties appropriate for local administration (e.g., tight joint spaces, intradisc spaces muscles (such as heart tissue), intra-arterial tissue, and the like) and exhibit significantly reduced injection force without compromising, and potentially even improving, the in vivo release profile of the beneficial agent.

At the end of study (i.e., at day 28), the depot gels were retrieved from the rats. Generally, a one-piece intact round-shaped depot was recovered corresponding to each injected depot in the animal.

The above-described exemplary embodiments are intended to be illustrative in all respects, rather than restrictive, of the present invention. Thus the present invention is capable of many variations in detailed implementation that can be derived from the description contained herein by a person skilled in the art. All such variations and modifications are considered to be within the scope and spirit of the present invention.

1. An implantable elastomeric depot composition for sustained delivery of a beneficial agent to a subject in a controlled manner over a predetermined duration of time after administration comprising:
an elastomeric viscous gel formulation comprising a bioerodible, biocompatible, elastomeric polymer and a solvent having a miscibility in water of less than or equal to 7 wt. % at 25° C., in an amount effective to plasticize the polymer and form a gel therewith; and
a beneficial agent dissolved or dispersed in the gel, wherein said beneficial agent is delivered over a duration equal to or greater than one month.

2. The implantable elastomeric depot composition of claim 1, wherein the polymer is selected from the group consisting of lactic acid, glycolic acid, caprolactone, p-dioxanone (PDO), trimethylene carbonate (TMC), a copolymer, terpolymer, and combinations and mixtures thereof, wherein glycolic acid is the predominant polymer and the polymer has a molecular weight ranging from about 3,000 to about 120,000.

3. The implantable elastomeric depot composition of claim 1, wherein said beneficial agent is a systemic agent.

4. The implantable elastomeric depot composition of claim 1, further including at least one of the following: a pore former; a solubility modulator for the beneficial agent; and an osmotic agent.

5. The implantable elastomeric depot composition of claim 1, wherein the elastomeric viscous gel further comprises a polymer selected from the group consisting of polylactides, polyglycolides, poly(caprolactone), polyanhydrides, polyamines, polyesteramides, polyorthoesters, polydioxanones, polyacetals, polyketals, polycarbonates, polyphosphoesters, polyorthocarbonates, polyphosphazenes, succinates, poly(malic acid), poly(amino acids), polyvinylpyrrolidone, polyethylene glycol, polyhydroxycellulose, polyphosphoesters, polysaccharides, chitin, chitosan, hyaluronic acid, p-dioxanone (PDO), trimethylene carbonate (TMC), poly(propylene fumarate), poly(orthoesters), polyphosphoester, and copolymers, terpolymers and mixtures thereof.

6. The implantable elastomeric depot composition of claim 1, wherein the solvent is selected from an aromatic alcohol having the structural formula (I)
Ar-(L)n-OH (I) (I)
in which Ar is a substituted or unsubstituted aryl or heteroaryl group, n is zero or 1, and L is a linking moiety; and a solvent selected from the group consisting of esters of aromatic acids, aromatic ketones, and mixtures thereof.

7. The implantable elastomeric depot composition of claim 1, wherein the solvent is selected from the aromatic alcohol, lower alkyl and aralkyl esters of aryl acids; aryl, aralkyl and lower alkyl ketones; and lower alkyl esters of citric acid.

8. The implantable elastomeric depot composition of claim 1, wherein the solvent is selected from benzyl alcohol, benzyl benzoate and ethyl benzoate.

9. The implantable elastomeric depot composition of claim 1, wherein the solvent has a miscibility in water of less than 5 wt. %.

10. The implantable elastomeric depot composition of claim 1, wherein the solvent has a miscibility in water of less than 3 wt. %.

11. The implantable elastomeric depot composition of claim 1, wherein the beneficial agent is selected from a drug, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents; anticoagulants, fibrinolytic agents, growth factors, antibodies, ocular drugs, and metabolites, analogs, derivatives, fragments, and purified, isolated, recombinant and chemically synthesized versions of these species.

12. The implantable elastomeric depot composition of claim 1, wherein the beneficial agent is in the form of particles dispersed or dissolved in the viscous gel.

13. The implantable elastomeric depot composition of claim 12, wherein the beneficial agent has an average particle size of from 0.1 to 250 microns.

14. The implantable elastomeric depot composition of claim 12, wherein the particles further comprise a component selected from the group consisting of a stabilizing agent, bulking agent, chelating agent and a buffering agent.

15. The implantable elastomeric depot composition of claim 1, wherein the polymer is a terpolymer of lactic acid, glycolic acid, and caprolactone, and wherein glycolic acid is the predominant component.

16. The implantable elastomeric depot composition of claim 1, wherein the polymer comprises a blend of polymers with different end groups.

17. The implantable elastomeric depot composition of claim 1, wherein the polymer has a lactic acid/glycolic acid ratio of 50:50 and the composition has a duration of delivery ranging from two days to about one month.

18. The implantable elastomeric depot composition of claim 1, wherein the polymer has a lactic acid/glycolic acid ratio of 65:35 and the composition has a duration of delivery of about two months.

19. The implantable elastomeric depot composition of claim 1, wherein the polymer has a lactic acid/glycolic acid ratio of 75:25 or a lactic acid/caprolactone ratio of 75:25 and the composition has a duration of delivery of about three months to about four months.

20. The implantable elastomeric depot composition of claim 1, wherein the polymer has a lactic acid/glycolic acid ratio of 85:15 the composition has a duration of delivery of about five months.

21. The implantable elastomeric depot composition of claim 1, wherein the depot composition has a terpolymer of caprolactone, glycolic acid, and lactic acid with glycolic acid being present in greater than 50 wt % and lactic acid being present in greater than 10 wt %, wherein the composition has a duration of delivery of about one month.

22. The implantable elastomeric depot composition of claim 1, wherein the polymer has a weight average molecular weight ranging from about 3,000 to about 10,000 as determined by gel permeation chromatography (GPC).

23. The implantable elastomeric depot composition of claim 1, wherein the polymer has a weight average molecular weight ranging from about 10,000 to about 30,000 as determined by gel permeation chromatography (GPC).

24. The implantable elastomeric depot composition of claim 1, wherein the polymer has a weight average molecular weight ranging from about 30,000 to about 250,000 as determined by gel permeation chromatography (GPC).

25. The implantable elastomeric depot composition of claim 1, wherein the elastomeric viscous gels have a glass transition temperature that is less than 37° C.

26. An implantable elastomeric depot composition for sustained systemic delivery of a beneficial agent to a subject in a controlled manner over a duration equal to or greater than one week after administration comprising:
an elastomeric viscous gel formulation comprising:
a bioerodible, biocompatible elastomeric polymer selected from the group consisting of poly(lactide-co-glycolide) copolymers (PLGA) and poly(caprolactone-co-lactic acid) (PCL-co-LA) having a comonomer lactic acid/glycolic acid ratio of from about 50:50 to about 100:0 and a lactic acid/caprolactone ratio of from about 25:75 to about 75:25; and
a solvent having a miscibility in water of less than or equal to 7 wt. % at 25° C., in an amount effective to plasticize the polymer and form a gel therewith; and
a beneficial agent dissolved or dispersed in the gel.

27. The implantable elastomeric depot composition of claim 26, wherein the polymer has a polymer solvent ratio of about 40:60 to about 65:35.

28. The implantable elastomeric depot composition of claim 26, wherein the beneficial agent is a systemic agent.

29. The implantable elastomeric depot composition of claim 26, further including at least one of the following: a pore former; a solubility modulator for the beneficial agent; and an osmotic agent.

30. The implantable elastomeric depot composition of claim 26, wherein the solvent is selected from an aromatic alcohol having the structural formula (I)
Ar-(L)n-OH (I)
in which Ar is a substituted or unsubstituted aryl or heteroaryl group, n is zero or 1, and L is a linking moiety; and a solvent selected from the group consisting of esters of aromatic acids, aromatic ketones, and mixtures thereof.

31. The implantable elastomeric depot composition of claim 26, wherein the solvent is selected from the aromatic alcohol, lower alkyl and aralkyl esters of aryl acids; aryl, aralkyl and lower alkyl ketones; and lower alkyl esters of citric acid.

32. The implantable elastomeric depot composition of claim 26, wherein the solvent is selected from benzyl alcohol, benzyl benzoate and ethyl benzoate.

33. The implantable elastomeric depot composition of claim 26, wherein the solvent has a miscibility in water of less than 3 wt. %.

34. The implantable elastomeric depot composition of claim 26, wherein the beneficial agent is selected from a drug, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents, antiproliferative agents, antimitotic agents, angiogenic agents, antipsychotic agents, central nervous system (CNS) agents; anticoagulants, fibrinolytic agents, growth factors, antibodies, ocular drugs, and metabolites, analogs, derivatives, fragments, and purified, isolated, recombinant and chemically synthesized versions of these species.

35. The implantable elastomeric depot composition of claim 26, wherein the beneficial agent is in the form of particles dispersed or dissolved in the viscous gel.

36. The implantable elastomeric depot composition of claim 26, wherein the beneficial agent particles have an average particle size of from 0.1 to 250 microns.

37. A kit for administration for sustained delivery of a beneficial agent to a subject in a controlled manner over a predetermined duration of time after administration comprising:
a bioerodible, biocompatible, elastomeric polymer, wherein the polymer is a glycolic acid-based polymer;
a solvent having a miscibility in water of less than or equal to 7 wt. % at 25° C., in an amount effective to plasticize the polymer and form a gel therewith;
a beneficial agent dissolved or dispersed in the gel; and
one or more of the following:
an emulsifying agent;
a pore former;
a solubility modulator for the beneficial agent; and
an osmotic agent;
wherein at least the beneficial agent is maintained separated from the solvent until the time of administration of the beneficial agent to a subject.

38. The kit of claim 37, wherein further comprising a metering device, a catheter, a pump, a syringe pump, or an autoinjector.

39. A method of administering a beneficial agent to a subject in a controlled manner, comprising:
administering the implantable elastomeric depot composition of claim 1; and
forming an implant at the site wherein the implant provides sustained release of the beneficial agent at the site.

40. The method of claim 39, wherein the beneficial agent is delivered systemically in a controlled manner over a duration equal to or greater than one week and up to one year after administration.

41. The method of claim 39, wherein the beneficial agent is delivered locally in a controlled manner over a duration equal to or greater than one week and up to one year after administration.

42. The method of claim 39, wherein the beneficial agent is injected from a standard hypodermic syringe through a needle, a catheter, or a trocar.

43. A method of making an implantable elastomeric depot composition for sustained delivery of a beneficial agent to a subject in a controlled manner over a predetermined duration of time after administration comprising:
providing an elastomeric viscous gel formulation comprising a bioerodible, biocompatible, elastomeric polymer and a solvent having a miscibility in water of less than or equal to 7 wt. % at 25° C., in an amount effective to plasticize the polymer and form a gel therewith; and
incorporating a beneficial agent into the elastomeric viscous gel formulation.

44. The method of claim 43, wherein the beneficial agent has an average particle size of from about 0.1 to about 250 microns.

45. The method of claim 43, wherein the beneficial agent is spray dried or freeze dried.
